(19) United States
(12) Patent Application Publication
(10) Pub. No.: US 2026/0043018 A1
(43) Pub. Date: Feb. 12, 2026

(54) NUCLEIC ACID PURIFICATION

(71) Applicant: Arcturus Therapeutics, Inc., San Diego, CA (US)

(72) Inventors: Timothy Wayne Luger, II, San Diego, CA (US); Maher Alayyoubi, San Diego, CA (US)

(21) Appl. No.: 19/292,296

(22) Filed: Aug. 6, 2025

Related U.S. Application Data
(60) Provisional application No. 63/680,505, filed on Aug. 7, 2024.

(51) Int. Cl. C12N 15/10; A61K 31/7088; A61K 39/00; C12P 19/34; C12Q 1/68

(52) U.S. Cl. CPC C12N 15/101; A61K 31/7088; A61K 39/00; C12P 19/34; C12Q 1/68; A61K 2039/53

(57) ABSTRACT

Disclosed herein are methods of purifying nucleic acids with reduced impurities. Some such methods include purifying transcribed RNA products, and may result in a reduction in dsRNA and other impurities compared to other methods.

### CROSS-REFERENCE TO RELATED APPLICATIONS

The present disclosure claims the benefit of priority from U.S. patent application No. 63/680,505, filed Aug. 7, 2024, the contents of which are incorporated herein by reference in their entirety.

### FIELD

The present disclosure generally relates to methods for purifying nucleic acids.

### BACKGROUND

RNA-based therapeutics and vaccines are at the frontier of modern medicine providing hope for those suffering from genetic diseases and those fearing deadly pathogens. For example, messenger RNA (mRNA) can be used in protein replacement therapy to treat diseases caused by a lack of protein, or by defective proteins, such as in cystic fibrosis. If a gene has a mutation that stops it from producing protein or causes it to produce defective protein, mRNA medicine can provide a healthy version of the missing protein.

RNA-based vaccines have emerged as a new class of RNA medicines. RNA vaccines can be developed more rapidly than traditional vaccines in response to infectious disease outbreaks as shown by the first two vaccines to obtain emergency use authorization from the FDA for the prevention of COVID-19, a deadly viral infection caused by SARS-COV-2.

One challenge to the development of RNA-based therapeutics and vaccines is the robust and efficient manufacture of mRNA with high yields and low levels of impurities, such as double-stranded RNA (dsRNA) and short abortive mRNA. Double-stranded RNA and short abortive mRNA are aberrant by-products of in vitro transcription (IVT) enzymatic reactions. Double-stranded RNA induces the immune response, inhibits protein translation, and hence decreases the safety/efficacy of the mRNA therapeutics and vaccines. Hence, there is an urgent and unmet need in manufacturing processes to address the removal of dsRNA either at the IVT level or in downstream purification steps. Further, there is an urgent need to develop mRNA manufacturing processes that reliably and efficiently produce mRNA with reduced dsRNA and short abortive mRNA impurities.

### SUMMARY

The present disclosure relates to methods for purifying nucleic acids such as RNA or mRNA to produce higher percentage full length transcripts. The nucleic acids may include in vitro transcription products. Described herein are methods of purifying transcribed nucleic acid (e.g., RNA) products with increased yield and reduced impurities.

In one aspect, provided is a method of purifying a nucleic acid comprising an RNA transcription product, comprising (a) providing an RNA transcription product composition comprising the RNA transcription product and having a conductivity of less than 20 mS/cm; (b) introducing the RNA transcription product composition to a polynucleotide affinity column; and (c) eluting the RNA transcription product from the column to obtain a purified RNA transcription product. In some embodiments, providing an RNA transcription product composition of step (a) comprises obtaining a crude RNA transcript composition from an in vitro transcription reaction and adjusting the conductivity of the crude RNA transcript composition to be less than 20 mS/cm. In some embodiments, adjusting the conductivity of the crude RNA transcript composition comprises diluting the crude RNA transcript composition with a conductivity decreasing agent. In some embodiments, the purified RNA transcription product has a purity of at least 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9%, and any sub value or subrange found within this range of numbers, inclusive of the endpoints.

In another aspect, provided is a method of purifying a nucleic acid, comprising obtaining a conductivity measurement of a composition comprising the nucleic acid; and when the conductivity measurement is at or below a threshold, introducing the composition to a polynucleotide affinity column.

In another aspect, provided is a method of purifying a nucleic acid, comprising obtaining a conductivity measurement of a composition comprising the nucleic acid; and when the conductivity measurement is at or below a threshold, enriching or purifying the nucleic acid from the composition.

In another aspect, provided is a method of purifying a nucleic acid, comprising obtaining a conductivity measurement of a composition comprising the nucleic acid; and when the conductivity measurement is at or below a threshold, formulating the nucleic acid of the composition into a pharmaceutical composition.

### BRIEF SUMMARY OF THE DRAWINGS

The following detailed description, given by way of example, but not intended to limit the invention solely to the specific embodiments described, may best be understood in conjunction with the accompanying drawings.

FIG. 1 illustrates a correlation between the conductivity of RNA #1 after dilution with water for injection, and the resulting purity of RNA #1 after purification by dT column, as described in Example 3.

FIG. 2 illustrates the improvement in percent purity of RNA #2 after purification by dT column, based on the conductivity of RNA #2 prior to purification, as described in Example 4.

FIG. 3 provides an exemplary fragment analyzer chromatogram of a purified nucleic acid, illustrating the delineations between the primary peak (P), the high molecular weight impurities (H), and low molecular weight impurities (L).

### DETAILED DESCRIPTION

### I. General

Disclosed herein are methods of purifying nucleic acids. In some embodiments, the nucleic acid is RNA, such as messenger RNA (mRNA), self-amplifying RNA (saRNA), circular RNA, trans-amplifying RNA, non-coding RNA (ncRNA), long non-coding RNA (InRNA), transfer RNA (tRNA), ribosomal RNA, miRNA, siRNA, antisense RNA, aptamers, and guide RNA. The method may include obtaining a conductivity measurement of a composition. Unless otherwise indicated, all conductivity values (e.g., in mS/cm or uS/cm) are values as measured at ambient temperature (i.e., room temperature). The composition may include nucleic acids or a nucleic acid. When the conductivity measurement is at or below a threshold, the method may include enriching or purifying the nucleic acid or nucleic acids. The enrichment or purification may include introducing the composition to a polynucleotide affinity column. The method may further include eluting the nucleic acid or nucleic acids from the affinity column. The nucleic acid or nucleic acids may include an RNA transcription product. An example threshold is 20 mS/cm or about 20 mS/cm. Some embodiments include a method of preparing a pharmaceutical composition comprising the nucleic acid or nucleic acids. In some such embodiments, when the conductivity measurement is at or below a threshold, the method includes formulating the nucleic acid or nucleic acids into a pharmaceutical composition.

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, figures and detailed description are to be regarded as illustrative in nature and not as restrictive.

### II. Methods of Purifying a Nucleic Acid Comprising an RNA Transcription Product

In one aspect, provided is a method of purifying a nucleic acid comprising an RNA transcription product, comprising:
(a) providing an RNA transcription product composition comprising the RNA transcription product and having a conductivity of less than 20 mS/cm;
(b) introducing the RNA transcription product composition to a polynucleotide affinity column; and
(c) eluting the RNA transcription product from the column to obtain a purified RNA transcription product.

In some embodiments, the conductivity of the RNA transcription product composition is less than 30 mS/cm, less than 25 mS/cm, less than 20 mS/cm, less than 19 mS/cm, less than 18 mS/cm, less than 17 mS/cm, less than 16 mS/cm, less than 15 mS/cm, less than 14 mS/cm, less than 13 mS/cm, less than 12 mS/cm, less than 11 mS/cm, less than 10 mS/cm, less than 9 mS/cm, less than 8 mS/cm, less than 7 mS/cm, less than 6 mS/cm, less than 5 mS/cm, less than 4 mS/cm, less than 3 mS/cm, less than 2 mS/cm, less than 1 mS/cm, less than 0.5 mS/cm, or any sub value or subrange between 19 mS/cm and 0.5 mS/cm, inclusive of endpoints. In some embodiments, the conductivity of the RNA transcription product composition is less than 19 mS/cm. In some embodiments, the conductivity of the RNA transcription product composition is less than 18 mS/cm. In some embodiments, the conductivity of the RNA transcription product composition is less than 17 mS/cm. In some embodiments, the conductivity of the RNA transcription product composition is less than 16 mS/cm. In some embodiments, the conductivity of the RNA transcription product composition is less than 15 mS/cm. In some embodiments, the conductivity of the RNA transcription product composition is less than 14 mS/cm. In some embodiments, the conductivity of the RNA transcription product composition is less than 13 mS/cm. In some embodiments, the conductivity of the RNA transcription product composition is less than 12 mS/cm. In some embodiments, the conductivity of the RNA transcription product composition is less than 11 mS/cm. In some embodiments, the conductivity of the RNA transcription product composition is less than 10 mS/cm. In some embodiments, the conductivity of the RNA transcription product composition is less than 9 mS/cm. In some embodiments, the conductivity of the RNA transcription product composition is less than 8 mS/cm. In some embodiments, the conductivity of the RNA transcription product composition is less than 5 mS/cm. In some embodiments, the conductivity of the RNA transcription product composition is less than 4 mS/cm. In some embodiments, the conductivity of the RNA transcription product composition is less than 3 mS/cm. In some embodiments, the conductivity of the RNA transcription product composition is 5 mS/cm or less. In some embodiments, the conductivity of the RNA transcription product composition is 4.28 mS/cm or less. In some embodiments, the conductivity of the RNA transcription product composition is 4 mS/cm or less. In some embodiments, the conductivity of the RNA transcription product composition is 3.67 mS/cm or less. In some embodiments, the conductivity of the RNA transcription product composition is 3.36 mS/cm or less. In some embodiments, the conductivity of the RNA transcription product composition is 2.22 mS/cm or less. In some embodiments, the conductivity of the RNA transcription product composition is less than 2 mS/cm. In some embodiments, the conductivity of the RNA transcription product composition is less than 1.7 mS/cm. In some embodiments, the conductivity of the RNA transcription product composition is less than 1.5 mS/cm.

In some embodiments, the conductivity of the RNA transcription product composition is about 0.5 mS/cm to about 20 mS/cm, or any sub value or subrange therebetween, inclusive of endpoints. In some embodiments, the conductivity of the RNA transcription product composition is about 0.5 mS/cm to about 18 mS/cm. In some embodiments, the conductivity of the RNA transcription product composition is about 0.5 mS/cm to about 16 mS/cm. In some embodiments, the conductivity of the RNA transcription product composition is about 0.5 mS/cm to about 14 mS/cm. In some embodiments, the conductivity of the RNA transcription product composition is about 0.5 mS/cm to about 12 mS/cm. In some embodiments, the conductivity of the RNA transcription product composition is about 0.5 mS/cm to about 10 mS/cm. In some embodiments, the conductivity of the RNA transcription product composition is about 0.5 mS/cm to about 8 mS/cm. In some embodiments, the conductivity of the RNA transcription product composition is about 1 mS/cm to about 10 mS/cm. In some embodiments, the conductivity of the RNA transcription product composition is about 1 mS/cm to about 15 mS/cm. In some embodiments, the conductivity of the RNA transcription product composition is about 1 mS/cm to about 8 mS/cm. In some embodiments, the conductivity of the RNA transcription product composition is about 1 mS/cm to about 6 mS/cm. In some embodiments, the conductivity of the RNA transcription product composition is about 1.5 mS/cm to about 8 mS/cm. In some embodiments, the conductivity of the RNA transcription product composition is about 1 mS/cm to about 5 mS/cm. In some embodiments, the conductivity of the RNA transcription product composition is about 2 mS/cm to about 8 mS/cm. In some embodiments, the conductivity of the RNA transcription product composition is about 0.97, 1.17, 1.46, 1.66, 1.74, 1.82, 1.97, 1.99, 2.22, 2.63, 3.36, 3.67, 3.95, 4.28, 5.26, or 8.42 mS/cm. In some embodiments, the conductivity of the RNA transcription product composition is about 4 mS/cm. In some embodiments, the conductivity of the RNA transcription product composition is about 3 mS/cm. In some embodiments, the conductivity of the RNA transcription product composition is about 4.28 mS/cm. In some embodiments, the conductivity of the RNA transcription product composition is about 3.67 mS/cm. In some embodiments, the conductivity of the RNA transcription product composition is about 3.36 mS/cm. In some embodiments, the conductivity of the RNA transcription product composition is about 2.22 mS/cm. In some embodiments, the conductivity of the RNA transcription product composition is about 5.26 mS/cm. In some embodiments, the conductivity of the RNA transcription product composition is about 1.74 mS/cm. In some embodiments, the conductivity of the RNA transcription product composition is about 1.46 mS/cm.

The purity of the purified RNA transcription product can be determined by the percentage of nucleic acid content determined by chromatography (e.g. using a fragment analyzer instrument) corresponding to the RNA product (e.g. primary peak (P) or primary peak and high molecular weight impurities (P+H)). In some instances, the purity of the purified product is determined by the percentage of nucleic acid content determined by chromatograph corresponding to the primary peak (P). In some instances, the purity of the purified product is determined by the percentage of nucleic acid content determined by chromatograph corresponding to the primary peak and the high molecular weight impurities (P+H). In some embodiments, the purity of the purified RNA transcription product is determined by fragment P+H, with P+H=integrated P (primary peak)+H (high molecular weight impurities), excluding L (low molecular weight impurities), wherein P+H+L=100%. In some embodiments, the purified RNA transcription product has a purity of at least 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9%, and any sub value or subrange found within this range of numbers, inclusive of the endpoints. In some embodiments, the purified RNA transcription product has a purity of at least 60%. In some embodiments, the purified RNA transcription product has a purity of at least 70%. In some embodiments, the purified RNA transcription product has a purity of at least 73%. In some embodiments, the purified RNA transcription product has a purity of at least 75%. In some embodiments, the purified RNA transcription product has a purity of at least 78%. In some embodiments, the purified RNA transcription product has a purity of at least 80%. In some embodiments, the purified RNA transcription product has a purity of at least 82%. In some embodiments, the purified RNA transcription product has a purity of at least 83%. In some embodiments, the purified RNA transcription product has a purity of at least 85%. In some embodiments, the purified RNA transcription product has a purity of at least 88%.

In some embodiments, the purified RNA transcription product has a purity of about 60% to about 99.9%. In some embodiments, the purified RNA transcription product has a purity of about 65% to about 99.9%. In some embodiments, the purified RNA transcription product has a purity of about 70% to about 99.9%. In some embodiments, the purified RNA transcription product has a purity of about 73% to about 99.9%. In some embodiments, the purified RNA transcription product has a purity of about 75% to about 99.9%. In some embodiments, the purified RNA transcription product has a purity of about 80% to about 99.9%. In some embodiments, the purified RNA transcription product has a purity of about 60% to about 95%. In some embodiments, the purified RNA transcription product has a purity of about 60% to about 90%. In some embodiments, the purified RNA transcription product has a purity of about 70% to about 95%. In some embodiments, the purified RNA transcription product has a purity of about 70% to about 90%. In some embodiments, the purified RNA transcription product has a purity of about 75% to about 90%. In some embodiments, the purified RNA transcription product has a purity of about 73% to about 90%. In some embodiments, the purified RNA transcription product has a purity of about 75% to about 90%. In some embodiments, the purified RNA transcription product has a purity of about 76% to about 90%. In some embodiments, the purified RNA transcription product has a purity of about 80% to about 90%.

In some embodiments, providing an RNA transcription product composition of step (a) comprises (a1) obtaining a crude RNA transcript composition from an in vitro transcription reaction and (a2) adjusting the conductivity of the crude RNA transcript composition to be less than 20 mS/cm. In some embodiments, (a2) adjusting the conductivity of the crude RNA transcript composition to be less than 20 mS/cm comprises adjusting the conductivity of the crude RNA transcript composition to be less than 19 mS/cm, less than 18 mS/cm, less than 17 mS/cm, less than 16 mS/cm, less than 15 mS/cm, less than 14 mS/cm, less than 13 mS/cm, less than 12 mS/cm, less than 11 mS/cm, less than 10 mS/cm, less than 9 mS/cm, less than 8 mS/cm, less than 7 mS/cm, less than 6 mS/cm, less than 5 mS/cm, less than 4 mS/cm, less than 3 mS/cm, less than 2 mS/cm, or less than 1 mS/cm. In some embodiments, the conductivity of the crude RNA transcript composition is adjusted to less than 19 mS/cm. In some embodiments, the conductivity of the crude RNA transcript composition is adjusted to less than 18 mS/cm. In some embodiments, the conductivity of the crude RNA transcript composition is adjusted to less than 17 mS/cm. In some embodiments, the conductivity of the crude RNA transcript composition is adjusted to less than 16 mS/cm. In some embodiments, the conductivity of the crude RNA transcript composition is adjusted to less than 15 mS/cm. In some embodiments, the conductivity of the crude RNA transcript composition is adjusted to less than 14 mS/cm. In some embodiments, the conductivity of the crude RNA transcript composition is adjusted to less than 13 mS/cm. In some embodiments, the conductivity of the crude RNA transcript composition is adjusted to less than 12 mS/cm. In some embodiments, the conductivity of the crude RNA transcript composition is adjusted to less than 11 mS/cm. In some embodiments, the conductivity of the crude RNA transcript composition is adjusted to less than 10 mS/cm. In some embodiments, the conductivity of the crude RNA transcript composition is adjusted to less than 9 mS/cm. In some embodiments, (a2) adjusting the conductivity of the crude RNA transcript composition to be less than 20 mS/cm comprises adjusting the conductivity of the crude RNA transcript composition to be less than 8 mS/cm. In some embodiments, (a2) adjusting the conductivity of the crude RNA transcript composition to be less than 20 mS/cm comprises adjusting the conductivity of the crude RNA transcript composition to be less than 5 mS/cm. In some embodiments, (a2) adjusting the conductivity of the crude RNA transcript composition to be less than 20 mS/cm comprises adjusting the conductivity of the crude RNA transcript composition to be less than 4 mS/cm. In some embodiments, (a2) adjusting the conductivity of the crude RNA transcript composition to be less than 20 mS/cm comprises adjusting the conductivity of the crude RNA transcript composition to be less than 3 mS/cm. In some embodiments, (a2) adjusting the conductivity of the crude RNA transcript composition to be less than 20 mS/cm comprises adjusting the conductivity of the crude RNA transcript composition to be 5 mS/cm or less. In some embodiments, (a2) adjusting the conductivity of the crude RNA transcript composition to be less than 20 mS/cm comprises adjusting the conductivity of the crude RNA transcript composition to be 4.28 mS/cm or less. In some embodiments, (a2) adjusting the conductivity of the crude RNA transcript composition to be less than 20 mS/cm comprises adjusting the conductivity of the crude RNA transcript composition to be 4 mS/cm or less. In some embodiments, (a2) adjusting the conductivity of the crude RNA transcript composition to be less than 20 mS/cm comprises adjusting the conductivity of the crude RNA transcript composition to be 3.67 mS/cm or less. In some embodiments, (a2) adjusting the conductivity of the crude RNA transcript composition to be less than 20 mS/cm comprises adjusting the conductivity of the crude RNA transcript composition to be 3.36 mS/cm or less. In some embodiments, (a2) adjusting the conductivity of the crude RNA transcript composition to be less than 20 mS/cm comprises adjusting the conductivity of the crude RNA transcript composition to be 2.22 mS/cm or less. In some embodiments, the conductivity of the crude RNA transcript composition is adjusted to less than 2 mS/cm. In some embodiments, the conductivity of the crude RNA transcript composition is adjusted to less than 1.7 mS/cm. In some embodiments, the conductivity of the crude RNA transcript composition is adjusted to less than 1.5 mS/cm. In some embodiments, the conductivity of the crude RNA transcript composition is adjusted to about 0.5 mS/cm to about 20 mS/cm. In some embodiments, the conductivity of the crude RNA transcript composition is adjusted to about 0.5 mS/cm to about 18 mS/cm. In some embodiments, the conductivity of the crude RNA transcript composition is adjusted to about 0.5 mS/cm to about 16 mS/cm. In some embodiments, the conductivity of the crude RNA transcript composition is adjusted to about 0.5 mS/cm to about 14 mS/cm. In some embodiments, the conductivity of the crude RNA transcript composition is adjusted to about 0.5 mS/cm to about 12 mS/cm. In some embodiments, the conductivity of the crude RNA transcript composition is adjusted to about 0.5 mS/cm to about 10 mS/cm. In some embodiments, the conductivity of the crude RNA transcript composition is adjusted to about 0.5 mS/cm to about 8 mS/cm. In some embodiments, the conductivity of the crude RNA transcript composition is adjusted to about 1 mS/cm to about 10 mS/cm. In some embodiments, the conductivity of the crude RNA transcript composition is adjusted to about 1 mS/cm to about 15 mS/cm. In some embodiments, the conductivity of the crude RNA transcript composition is adjusted to about 1 mS/cm to about 8 mS/cm. In some embodiments, the conductivity of the crude RNA transcript composition is adjusted to about 1 mS/cm to about 6 mS/cm. In some embodiments, the conductivity of the crude RNA transcript composition is adjusted to about 1.5 mS/cm to about 8 mS/cm. In some embodiments, the conductivity of the crude RNA transcript composition is adjusted to about 1 mS/cm to about 5 mS/cm. In some embodiments, the conductivity of the crude RNA transcript composition is adjusted to about 2 mS/cm to about 8 mS/cm. In some embodiments, the conductivity of the crude RNA transcript composition is adjusted to about 0.97, 1.17, 1.46, 1.66, 1.74, 1.82, 1.97, 1.99, 2.22, 2.63, 3.36, 3.67, 3.95, 4.28, 5.26, or 8.42 mS/cm, and any sub value or subrange found within this range of numbers, inclusive of the endpoints. In some embodiments, the conductivity of the crude RNA transcript composition is adjusted to about 5.26 mS/cm. In some embodiments, the conductivity of the crude RNA transcript composition is adjusted to about 1.74 mS/cm. In some embodiments, the conductivity of the crude RNA transcript composition is adjusted to about 1.46 mS/cm. In some embodiments, (a2) adjusting the conductivity of the crude RNA transcript composition to be less than 20 mS/cm comprises diluting the crude RNA transcript composition with a conductivity decreasing agent.

In some embodiments, the conductivity decreasing agent has a conductivity less than 2 mS/cm. In some embodiments, the conductivity decreasing agent has a conductivity less than 1 mS/cm. In some embodiments, the conductivity decreasing agent has a conductivity of about 0 mS/cm. In some embodiments, the conductivity decreasing agent has a conductivity of less than about 100 μS/cm. In some embodiments, the conductivity decreasing agent has a conductivity of less than about 50 μS/cm. In some embodiments, the conductivity decreasing agent has a conductivity of less than about 10 μS/cm. In some embodiments, the conductivity decreasing agent has a conductivity of less than about 5 μS/cm.

In some embodiments, providing an RNA transcription product composition of step (a) comprises obtaining a crude RNA transcript composition from an in vitro transcription reaction and adjusting the conductivity of the crude RNA transcript composition to 5 mS/cm or lower; and the purified RNA transcription product has a purity of at least 73%. In some embodiments, providing an RNA transcription product composition of step (a) comprises obtaining a crude RNA transcript composition from an in vitro transcription reaction and adjusting the conductivity of the crude RNA transcript composition to 4.28 mS/cm or lower; and the purified RNA transcription product has a purity of at least 73%. In some embodiments, providing an RNA transcription product composition of step (a) comprises obtaining a crude RNA transcript composition from an in vitro transcription reaction and adjusting the conductivity of the crude RNA transcript composition to 4 mS/cm or lower; and the purified RNA transcription product has a purity of at least 75%. In some embodiments, providing an RNA transcription product composition of step (a) comprises obtaining a crude RNA transcript composition from an in vitro transcription reaction and adjusting the conductivity of the crude RNA transcript composition to be 3.36 mS/cm or lower; and the purified RNA transcription product has a purity of at least 75%. In some embodiments, providing an RNA transcription product composition of step (a) comprises obtaining a crude RNA transcript composition from an in vitro transcription reaction and adjusting the conductivity of the crude RNA transcript composition to be about 1 mS/cm to about 2 mS/cm; and the purified RNA transcription product has a purity of about 75% to about 95%. In some embodiments, providing an RNA transcription product composition of step (a) comprises obtaining a crude RNA transcript composition from an in vitro transcription reaction and adjusting the conductivity of the crude RNA transcript composition to be about 1 mS/cm to about 8 mS/cm; and the purified RNA transcription product has a purity of about 70% to about 95%.

### III. Further Methods of Purifying a Nucleic Acid

In one aspect, provided is a method of purifying a nucleic acid, comprising: obtaining a conductivity measurement of a composition comprising the nucleic acid; and when the conductivity measurement is at or below a threshold, introducing the composition to a polynucleotide affinity column. In some embodiments, the method comprises: when the conductivity measurement is not at or below a threshold, not introducing the composition to a polynucleotide affinity column. In some embodiments, the method comprises: when the conductivity measurement is not at or below a threshold, adjusting the conductivity of the composition to be at or below the threshold. In some embodiments, the method comprises: introducing the composition to the polynucleotide affinity column after adjusting the conductivity of the composition to be at or below the threshold.

In another aspect, provided is a method of purifying a nucleic acid, comprising: obtaining a conductivity measurement of a composition comprising the nucleic acid; and when the conductivity measurement is at or below a threshold, enriching or purifying the nucleic acid from the composition. In some embodiments, the method comprises: when the conductivity measurement is not at or below a threshold, not enriching or purifying the nucleic acid from the composition. In some embodiments, the method comprises: when the conductivity measurement is not at or below a threshold, adjusting the conductivity of the composition to be at or below the threshold. In some embodiments, the method comprises: enriching or purifying the nucleic acid from the composition after adjusting the conductivity of the composition to be at or below the threshold.

In another aspect, provided is a method of preparing a pharmaceutical composition comprising a nucleic acid, the method comprising: obtaining a conductivity measurement of a composition comprising the nucleic acid; and when the conductivity measurement is at or below a threshold, formulating the nucleic acid of the composition into the pharmaceutical composition. In some embodiments, the method comprises: when the conductivity measurement is not at or below a threshold, not formulating the nucleic acid of the composition into the pharmaceutical composition. In some embodiments, the method comprises: when the conductivity measurement is not at or below a threshold, adjusting the conductivity of the composition to be at or below the threshold. In some embodiments, the method comprises: formulating the nucleic acid of the composition into the pharmaceutical composition after adjusting the conductivity of the composition to be at or below the threshold. The threshold for the conductivity of the composition such as the RNA transcription product composition can be as described herein. In some embodiments, the method comprises: enriching or purifying the nucleic acid prior to formulating the nucleic acid of the composition into the pharmaceutical composition. In some embodiments, the pharmaceutical composition comprises a lipid or nanoparticle. In some embodiments, the pharmaceutical composition comprises a solid lipid nanoparticle. In some embodiments, the pharmaceutical composition is a vaccine. In some embodiments, the method further comprises translating the nucleic acid to a protein. In some embodiments, the translating is in vivo.

In some embodiments, the composition comprising the nucleic acid is an RNA transcription product composition. In some embodiments, the RNA transcription product composition is from an in vitro transcription reaction. In some embodiments, the RNA transcription product composition comprises a DNase, ethylenediaminetetraacetic acid (EDTA), free nucleotides, a buffer, and an RNA. In some embodiments, the RNA transcription product composition comprises an RNAse inhibitor, pyrophosphatase, buffer components, dsRNA, short abortive mRNA, pyrophosphate, phosphate, a DNAse, template DNA fragments, free nucleotides, ethylenediaminetetraacetic acid (EDTA), free nucleotides, a buffer, and an RNA.

In some embodiments, the nucleic acid comprises a transcription product. In some embodiments, the nucleic acid comprises an RNA. In some embodiments, the RNA comprises a replicon. In some embodiments, the RNA comprises an mRNA. In some embodiments, the mRNA comprises a poly-A tail.

In some embodiments, the method further comprises providing or obtaining the composition comprising the nucleic acid. In some embodiments, providing or obtaining the composition comprises performing in vitro transcription.

In some embodiments, the composition comprising the nucleic acid is formulated for loading onto a purification column. In some embodiments, the composition comprising the nucleic acid is formulated for loading onto the polynucleotide affinity column.

In some embodiments, obtaining the conductivity measurement of the composition comprising the nucleic acid comprises determining an ability of the composition to conduct electricity. In some embodiments, obtaining the conductivity measurement of the composition comprising the nucleic acid comprises determining a resistance of the composition between electrodes.

In some embodiments, the threshold is less than 30 mS/cm, less than 29 mS/cm, less than 28 mS/cm, less than 27 mS/cm, less than 26 mS/cm, less than 25 mS/cm, less than 24 mS/cm, less than 23 mS/cm, less than 22 mS/cm, less than 21 mS/cm, less than 20 mS/cm, less than 19 mS/cm, less than 18 mS/cm, less than 17 mS/cm, less than 16 mS/cm, less than 15 mS/cm, less than 14 mS/cm, less than 13 mS/cm, less than 12 mS/cm, less than 11 mS/cm, less than 10 mS/cm, less than 9 mS/cm, less than 8 mS/cm, less than 7 mS/cm, less than 6 mS/cm, less than 5 mS/cm, less than 4 mS/cm, less than 3 mS/cm, less than 2 mS/cm, or less than 1 mS/cm. In some embodiments, the threshold is less than 30 mS/cm. In some embodiments, the threshold is less than 27 mS/cm. In some embodiments, the threshold is less than 25 mS/cm. In some embodiments, the threshold is less than 23 mS/cm. In some embodiments, the threshold is less than 22 mS/cm. In some embodiments, the threshold is less than 20 mS/cm. In some embodiments, the threshold is less than 19 mS/cm. In some embodiments, the threshold is less than 17 mS/cm. In some embodiments, the threshold is less than 15 mS/cm. In some embodiments, the threshold is less than 13 mS/cm. In some embodiments, the threshold is less than 10 mS/cm. In some embodiments, the threshold is less than 8 mS/cm. In some embodiments, the threshold is less than 5 mS/cm. In some embodiments, the threshold is less than 4 mS/cm. In some embodiments, the threshold is less than 3 mS/cm. In some embodiments, the threshold is less than 4.28 mS/cm. In some embodiments, the threshold is less than 3.67 mS/cm. In some embodiments, the threshold is less than 3.36 mS/cm. In some embodiments, the threshold is less than 2.22 mS/cm.

In some embodiments, the threshold is about 1 mS/cm to about 25 mS/cm. In some embodiments, the threshold is about 1 mS/cm to about 20 mS/cm. In some embodiments, the threshold is about 1 mS/cm to about 18 mS/cm. In some embodiments, the threshold is about 1 mS/cm to about 16 mS/cm. In some embodiments, the threshold is about 1 mS/cm to about 15 mS/cm. In some embodiments, the threshold is about 1 mS/cm to about 14 mS/cm. In some embodiments, the threshold is about 1 mS/cm to about 13 mS/cm. In some embodiments, the threshold is about 1 mS/cm to about 12 mS/cm. In some embodiments, the threshold is about 1 mS/cm to about 11 mS/cm. In some embodiments, the threshold is about 1 mS/cm to about 10 mS/cm. In some embodiments, the threshold is about 1 mS/cm to about 9 mS/cm. In some embodiments, the threshold is about 1 mS/cm to about 8 mS/cm. In some embodiments, the threshold is about 1 mS/cm to about 7 mS/cm. In some embodiments, the threshold is about 1 mS/cm to about 6 mS/cm. In some embodiments, the threshold is about 1 mS/cm to about 5 mS/cm. In some embodiments, the threshold is about 1 mS/cm to about 4 mS/cm. In some embodiments, the threshold is about 1 mS/cm to about 3 mS/cm. In some embodiments, the threshold is about 1.5 mS/cm to about 8 mS/cm. In some embodiments, the threshold is about 2 mS/cm to about 8 mS/cm.

In some embodiments, the threshold is about 30 mS/cm, about 29 mS/cm, about 28 mS/cm, about 27 mS/cm, about 26 mS/cm, about 25 mS/cm, about 24 mS/cm, about 23 mS/cm, about 22 mS/cm, about 21 mS/cm, about 20 mS/cm, about 19 mS/cm, about 18 mS/cm, about 17 mS/cm, about 16 mS/cm, about 15 mS/cm, about 14 mS/cm, about 13 mS/cm, about 12 mS/cm, about 11 mS/cm, about 10 mS/cm, about 9 mS/cm, about 8 mS/cm, about 7 mS/cm, about 6 mS/cm, about 5 mS/cm, about 4 mS/cm, about 3 mS/cm, about 2 mS/cm, or about 1 mS/cm. In some embodiments, the threshold is 20 mS/cm. In some embodiments, the threshold is 8 mS/cm. In some embodiments, the threshold is 5 mS/cm. In some embodiments, the threshold is about 0.97, 1.17, 1.46, 1.66, 1.74, 1.82, 1.97, 1.99, 2.22, 2.63, 3.36, 3.67, 3.95, 4.28, 5.26, or 8.42 mS/cm. In some embodiments, the threshold is 4 mS/cm. In some embodiments, the threshold is 3 mS/cm. In some embodiments, the threshold is 4.28 mS/cm. In some embodiments, the threshold is 3.67 mS/cm. In some embodiments, the threshold is 3.36 mS/cm. In some embodiments, the threshold is 2.22 mS/cm. In some embodiments, the threshold is about 5.26 mS/cm. In some embodiments, the threshold is about 1.74 mS/cm. In some embodiments, the threshold is about 1.46 mS/cm.

In some embodiments, adjusting the conductivity of the composition comprises diluting the composition. In some embodiments, adjusting the conductivity of the composition comprises diluting the composition with a conductivity decreasing agent. In some embodiments, diluting the composition comprises diluting the composition by a factor of about 1.125×, about 1.25×, about 1.5×, about 1.75×, about 2×, about 2.5×, about 3×, about 4×, about 5×, about 6×, about 7×, about 8×, about 9×, about 10×, about 11×, about 12×, about 13×, about 14×, or about 15×, or more, or a range defined by any two of the aforementioned factors, and any sub value or subrange found within this range of numbers, inclusive of the endpoints. For example, the dilution factor can be about 1.125×. In some embodiments, the dilution factor is about 1.5×. In some embodiments, the dilution factor is about 2×. In some embodiments, the dilution factor is about 3×. In some embodiments, the dilution factor is about 4×. In some embodiments, the dilution factor is about 4×. In some embodiments, the dilution factor is about 5×. In some embodiments, the dilution factor is about 6×. In some embodiments, the dilution factor is about 7×. In some embodiments, the dilution factor is about 8×. In some embodiments, the dilution factor is about 9×. In some embodiments, the dilution factor is about 10×. In some embodiments, the dilution factor is about 11×. In some embodiments, the dilution factor is about 12×. In some embodiments, the dilution factor is about 13×. In some embodiments, the dilution factor is about 14×. In some embodiments, diluting the composition comprises diluting the composition by a factor of 15×. In some embodiments, dilution the composition comprises diluting the composition by a factor of about 1.125× to about 20×, about 1.125× to about 18×, about 1.125× to about 17×, about 1.5× to about 17×, about 2× to about 17×, about 5× to about 17×, about 8× to about 17×, about 10× to about 17×, about 13× to about 17×, or about 10× to about 15×. In some embodiments, the dilution factor is about 5× to about 17×. In some embodiments, the dilution factor is about 7× to about 17×. In some embodiments, the dilution factor is about 8× to about 17×. In some embodiments, the dilution factor is about 10× to about 17×. In some embodiments, the dilution factor is about 13× to about 17×.

In some embodiments, the conductivity decreasing agent comprises water. In some embodiments, the conductivity decreasing agent comprises a low-conductivity solution. In some embodiments, the conductivity decreasing agent comprises water, ethanol, dimethylsulfoxide, urea or combinations thereof. In some embodiments, the conductivity decreasing agent comprises urea. In some embodiments, the conductivity decreasing agent comprises an organic solvent. In some embodiments, the conductivity decreasing agent comprises a water-miscible organic solvent. In some embodiments, the conductivity decreasing agent comprises ethanol or dimethylsulfoxide (DMSO). In some embodiments, the conductivity decreasing agent comprises DMSO. In some embodiments, the conductivity decreasing agent comprises ethanol. In some embodiments, the conductivity decreasing agent is 20% ethanol. In some embodiments, the conductivity decreasing agent is ultrapure water. In some embodiments, the conductivity decreasing agent is sterile. In some embodiments, the conductivity decreasing agent is Sartorius water. In some embodiments, the conductivity decreasing agent is formulated for an injection. In some embodiments, the conductivity decreasing agent is water for injection (WFI). In some embodiments, the conductivity decreasing agent is a urea solution. In some embodiments, the conductivity decreasing agent is a urea solution. In some embodiments, the conductivity decreasing agent is an aqueous urea solution. In some embodiments, the conductivity decreasing agent is an aqueous urea solution of about 1M. In some embodiments, the conductivity decreasing agent is an aqueous urea solution of about 2M. In some embodiments, the conductivity decreasing agent is an aqueous urea solution of about 3M. In some embodiments, the conductivity decreasing agent is an aqueous urea solution of about 4M. In some embodiments, the conductivity decreasing agent is an aqueous urea solution of about 5M. In some embodiments, the conductivity decreasing agent is an aqueous urea solution of about 6M. In some embodiments, the conductivity decreasing agent is an aqueous urea solution of about 7M. In some embodiments, the conductivity decreasing agent is an aqueous urea solution of about 8M. In some embodiments, the conductivity decreasing agent is an aqueous urea solution of about 9M. In some embodiments, the conductivity decreasing agent is an aqueous urea solution of about 10M. In some embodiments, the conductivity decreasing agent is an aqueous urea solution of about 0.5M to about 10M, about 1M to about 10M, about 1M to about 8M, about 1M to about 7M, about 1M to about 6M, about 2M to about 9M, about 3M to about 9M, about 3M to about 8M, about 4M to about 7M, or about 5M to about 7M, and any sub value or subrange found within this range of numbers, inclusive of the endpoints. In some embodiments, the aqueous urea solution is a buffered solution. In some embodiments, the buffer is a Tris buffer or a sodium phosphate buffer. In some embodiments, the buffer is a Tris buffer or sodium phosphate buffer of about 25 mM to about 500 mM. In some embodiments, the buffer is of pH of about 6.5 to about 8.5. In some embodiments, the buffer is of pH of about 7.0 to about 8.0. In some embodiments, the buffer is of pH of about 7.2 to about 7.7. In some embodiments, the buffer is of pH of about 7.5 to about 7.6. In some embodiments, the buffer is a Tris buffer. In some embodiments, the buffer is about 50 mM to about 1200 mM Tris. In some embodiments, the buffer is about 100 mM to about 500 mM Tris. In some embodiments, the buffer is about 300 mM to 500 mM or about 400 mM Tris. In some embodiments, the buffer is about 400 mM to about 1200 mM Tris. In some embodiments, the buffer is about 500 mM to 1100 mM or about 1000 mM Tris. In some embodiments, the buffer is 50 mM Tris, pH 7.5. In some embodiments, the buffer is sodium phosphate buffer of about 25 mM to about 500 mM. In some embodiments, the buffer is sodium phosphate buffer of about 30 mM to about 300 mM. In some embodiments, the buffer is sodium phosphate buffer of about 30 mM to about 150 mM. In some embodiments, the buffer is sodium phosphate buffer of about 30 mM to about 100 mM. In some embodiments, the buffer is sodium phosphate buffer of about 30 mM to about 70 mM, or about 50 mM. In some embodiments, the buffer is 50 mM sodium phosphate, pH 7.5 to 7.6.

In some embodiments, the conductivity decreasing agent has a conductivity of less than 20 mS/cm, less than 19 mS/cm, less than 18 mS/cm, less than 17 mS/cm, less than 16 mS/cm, less than 15 mS/cm, less than 14 mS/cm, less than 13 mS/cm, less than 12 mS/cm, less than 11 mS/cm, less than 10 mS/cm, less than 9 mS/cm, less than 8 mS/cm, less than 7 mS/cm, less than 6 mS/cm, less than 5 mS/cm, less than 4 mS/cm, less than 3 mS/cm, less than 2 mS/cm, or less than 1 mS/cm. In some embodiments, the conductivity decreasing agent has a conductivity of less than 19 mS/cm. In some embodiments, the conductivity decreasing agent has a conductivity of less than 18 mS/cm. In some embodiments, the conductivity decreasing agent has a conductivity of less than 17 mS/cm. In some embodiments, the conductivity decreasing agent has a conductivity of less than 15 mS/cm. In some embodiments, the conductivity decreasing agent has a conductivity of less than 13 mS/cm. In some embodiments, the conductivity decreasing agent has a conductivity of less than 10 mS/cm. In some embodiments, the conductivity decreasing agent has a conductivity of less than 8 mS/cm. In some embodiments, the conductivity decreasing agent has a conductivity of less than 6 mS/cm. In some embodiments, the conductivity decreasing agent has a conductivity of less than 5 mS/cm. In some embodiments, the conductivity decreasing agent has a conductivity less than 1 mS/cm. In some embodiments, the conductivity decreasing agent has a conductivity of less than about 500 μS/cm. In some embodiments, the conductivity decreasing agent has a conductivity of less than about 300 μS/cm. In some embodiments, the conductivity decreasing agent has a conductivity of less than about 100 μS/cm. In some embodiments, the conductivity decreasing agent has a conductivity of less than about 50 μS/cm. In some embodiments, the conductivity decreasing agent has a conductivity of less than about 30 μS/cm. In some embodiments, the conductivity decreasing agent has a conductivity of less than about 10 μS/cm. In some embodiments, the conductivity decreasing agent has a conductivity of less than about 5 μS/cm.

In some embodiments, the conductivity decreasing agent has a conductivity of about 20 mS/cm, of about 19 mS/cm, of about 18 mS/cm, of about 17 mS/cm, of about 16 mS/cm, of about 15 mS/cm, of about 14 mS/cm, of about 13 mS/cm, of about 12 mS/cm, of about 11 mS/cm, of about 10 mS/cm, of about 9 mS/cm, of about 8 mS/cm, of about 7 mS/cm, of about 6 mS/cm, of about 5 mS/cm, of about 4 mS/cm, of about 3 mS/cm, of about 2 mS/cm, or of about 1 mS/cm. In some embodiments, the conductivity decreasing agent has a conductivity of about 6 mS/cm. In some embodiments, the conductivity decreasing agent has a conductivity of about 5 mS/cm. In some embodiments, the conductivity decreasing agent has a conductivity of about 4 mS/cm. In some embodiments, the conductivity decreasing agent has a conductivity of about 3 mS/cm. In some embodiments, the conductivity decreasing agent has a conductivity of about 2 mS/cm. In some embodiments, the conductivity decreasing agent has a conductivity of about 1 mS/cm. In some embodiments, the conductivity decreasing agent has a conductivity of about 500 μS/cm. In some embodiments, the conductivity decreasing agent has a conductivity of about 300 μS/cm. In some embodiments, the conductivity decreasing agent has a conductivity of about 200 μS/cm. In some embodiments, the conductivity decreasing agent has a conductivity of about 100 μS/cm. In some embodiments, the conductivity decreasing agent has a conductivity of about 50 μS/cm. In some embodiments, the conductivity decreasing agent has a conductivity of about 5 μS/cm to about 1000 μS/cm. In some embodiments, the conductivity decreasing agent has a conductivity of about 50 μS/cm to about 800 μS/cm. In some embodiments, the conductivity decreasing agent has a conductivity of about 500 μS/cm to about 10 mS/cm. In some embodiments, the conductivity decreasing agent has a conductivity of about 500 μS/cm to about 8 mS/cm. In some embodiments, the conductivity decreasing agent has a conductivity of about 500 μS/cm to about 5 mS/cm. In some embodiments, the conductivity decreasing agent has a conductivity of about 500 μS/cm to about 4 mS/cm. In some embodiments, the conductivity decreasing agent has a conductivity of about 500 μS/cm to about 3 mS/cm. In some embodiments, the conductivity decreasing agent has a conductivity of about 500 μS/cm to about 1 mS/cm. In some embodiments, the conductivity decreasing agent has a conductivity of about 10 μS/cm to about 100 μS/cm. In some embodiments, the conductivity decreasing agent has a conductivity of about 10 μS/cm to about 50 μS/cm. In some embodiments, the conductivity decreasing agent has a conductivity of about 10 μS/cm to about 30 μS/cm. In some embodiments, the conductivity decreasing agent has a conductivity of about 10 μS/cm to about 300 μS/cm. In some embodiments, the conductivity decreasing agent has a conductivity of about 10 μS/cm to about 400 μS/cm. In some embodiments, the conductivity decreasing agent has a conductivity of about 20 μS/cm to about 400 μS/cm. In some embodiments, the conductivity decreasing agent has a conductivity of about 150 μS/cm to about 350 μS/cm. In some embodiments, the conductivity decreasing agent has a conductivity of about 200 μS/cm to about 300 μS/cm. In some embodiments, the conductivity decreasing agent has a conductivity of about 500 μS/cm to about 1.5 mS/cm. In some embodiments, the conductivity decreasing agent has a conductivity of about 800 μS/cm to about 1.3 mS/cm. In some embodiments, the conductivity decreasing agent has a conductivity of about 0 mS/cm. In some embodiments, the conductivity decreasing agent is or comprises ultrapure water, urea, or a water-miscible organic solvent such as ethanol, methanol, DMSO, or the like. In some embodiments, the conductivity decreasing agent is or comprises water, such as ultrapure water. In some embodiments, the conductivity decreasing agent is or comprises urea. In some embodiments, the conductivity decreasing agent is a 6M aqueous urea solution and when prepared fresh, has a conductivity of about 15 μS/cm to about 30 μS/cm, for example of about 21.12 μS/cm. It is understood that as a conductivity decreasing agent is stored, the conductivity may change slightly overtime. However, the conductivity of the conductivity decreasing agent may still fall within the ranges of conductivity as described here and the conductivity decreasing agent can still be suitable for the methods of the present disclosure. For example, an 8M aqueous urea solution when prepared fresh has a conductivity of about 235.9 μS/cm. After storage, the conductivity can increase to 1.025 mS/cm. Both the fresh aqueous urea solution and the stored urea solution can be used for the methods of the present disclosure.

In some embodiments, enriching or purifying the nucleic acid from the composition comprises performing affinity chromatography on the composition. In some embodiments, performing affinity chromatography on the composition comprises introducing the composition to a polynucleotide affinity column. In some embodiments, the polynucleotide affinity column comprises a poly-T (e.g. dT) column. In some embodiments, the polynucleotide affinity column comprises a solid support. In some embodiments, the solid support comprises a matrix, gel, silica, or bead. In some embodiments, introducing the composition to the polynucleotide affinity column comprises binding the nucleic acid to a component within the column. In some embodiments, the method further comprises performing a wash on the column while the nucleic acid is bound to a component within the column. In some embodiments, the method further comprises eluting the nucleic acid from the column. In some embodiments, the eluted, purified, or enriched nucleic acid is at least 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% free of contaminants. In some embodiments, the eluted, purified, or enriched nucleic acid is at least 70% free of contaminants. In some embodiments, the eluted, purified, or enriched nucleic acid is at least 75% free of contaminants. The contaminants can include low molecular weight impurities (L), residual proteins, residual DNA or other unwanted nucleic acid species. For example, the percentage of purity can be assessed using chromatography and optionally expressed as percentage of the primary peak (P).

In some embodiments, prior to obtaining the conductivity measurement, the nucleic acid or composition comprising the nucleic acid has undergone an additional enrichment or purification process. In some embodiments, the method further comprises performing an enrichment or purification process prior to obtaining the conductivity measurement. In some embodiments, the additional enrichment or purification process comprises affinity chromatography, high-performance liquid chromatography (e.g. C4), silica purification, or ultrafiltration and diafiltration (UFDF), or a combination thereof. In some embodiments, the purification process comprises silica purification.

In some embodiments, prior to obtaining the conductivity measurement, the composition or nucleic acid has not undergone any additional enrichment or purification.

In some embodiments, the method further comprises performing an additional enrichment or purification process on the composition or nucleic acid after introducing the composition to the polynucleotide affinity column, or after eluting the nucleic acid from the column. In some embodiments, the additional enrichment or purification process comprises affinity chromatography, high-performance liquid chromatography (e.g. C4), silica purification, or ultrafiltration and diafiltration (UFDF), or a combination thereof.

In some embodiments, the composition or nucleic acid does not undergo an additional enrichment or purification process after being introduced to the polynucleotide affinity column, or after being eluted from the column.

### IV. Definitions

Unless defined otherwise, all technical and scientific terms used herein have the same meaning as commonly understood by one of ordinary skill in the art to which this disclosure belongs. In this specification and in the claims that follow, reference will be made to a number of terms that shall be defined to have the following meanings:

The terminology used herein is for the purpose of describing particular embodiments only and is not intended to be limiting. As used herein, the singular forms “a”, “an” and “the” are intended to include the plural forms as well, unless the context clearly indicates otherwise.

“Optional” or “optionally” means that the subsequently described event or circumstance can or cannot occur, and that the description includes instances where the event or circumstance occurs and instances where it does not.

The term “about” when used before a numerical designation, e.g., temperature, time, amount, concentration, and such other, including a range, indicates approximations which may vary by (+) or (−) 10%, 5%, 1%, or any subrange or subvalue there between. Preferably, the term “about” when used with regard to an amount means that the amount may vary by +/−10%.

“Comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this disclosure.

The term “isolated,” “isolating,” “purified,” and the like, when applied to a nucleic acid, denotes that the nucleic acid is essentially free of other cellular components with which it is associated in the natural state. It can be, for example, in a homogeneous state and may be in either a dry or aqueous solution. Homogeneity is typically determined using analytical chemistry techniques such as polyacrylamide gel electrophoresis or high performance liquid chromatography. A nucleic acid that is the predominant species present in a preparation is substantially purified.

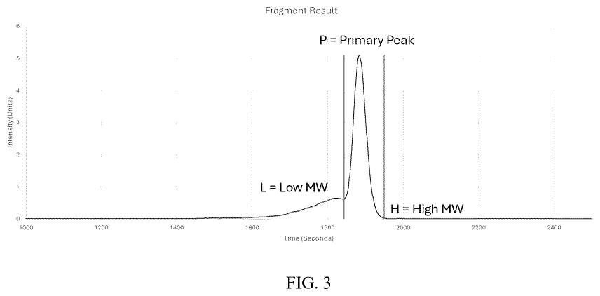

The “purity” or “% full length transcript” or “percent purity” of a nucleic acid with respect to the methods disclosed herein is determined as follows. A chromatogram is collected, and the resulting chromatographic trace is divided into three components: the primary peak (P), the high molecular weight impurities (H), and low molecular weight impurities (L). The maximum intensity peak of the chromatogram is assigned as P. To assign L and H, a 1st derivative is applied to each point on the chromatogram, and the maximum and minimum 1st derivatives closest to P are used to assign cutoff of L and H values, as illustrated in FIG. 3. Each component is integrated, and the purity is calculated as (P+H)/(P+H+L), wherein P+H+L=100%. In some embodiments, the chromatogram is collected using capillary electrophoresis. In some embodiments, the chromatogram is collected using a fragment analyzer (e.g., as sold by Agilent). In some embodiments, the chromatogram is collected using an analytical capillary electrophoresis instrument (e.g., as sold by SciEx). In some embodiments, the chromatogram is an electropherogram. In some embodiments, the chromatogram is collected using high performance liquid chromatography.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T), or uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

The term “transcription” as used herein, generally refers to the process of copying a segment of DNA into RNA, where the segments of DNA transcribed into RNA molecules that encode proteins produce mRNA. In embodiments, the segments of DNA that are copied into RNA molecules are referred to as non-coding RNAs.

The term “polymerase” as used herein, generally refers to an enzyme that catalyzes the synthesis of DNA or RNA whose sequence is complementary to the original template. In embodiments, RNA polymerase is the enzyme that synthesizes RNA from a DNA template.

The term “template” as used herein, generally refers to the antisense DNA strand. In embodiments, the cell uses the antisense strand as a template for producing messenger RNA (mRNA) that directs the synthesis of a protein. In embodiments, the term “linear DNA (L.DNA) template” generally refers to DNA antisense strands that have been uncoiled or linearized by the use of restriction enzyme or are PCR amplicons.

The term “DNase” as used herein, generally refers to deoxyribonuclease, which is an enzyme that catalyzes the hydrolytic cleavage of phosphodiester bonds that connect/link nucleotides in the DNA backbone.

As used herein, “transcribed RNA product,” “in vitro transcribed RNA,” “in vitro-synthesized RNA” and the like generally refers to mRNA that is synthesized or prepared using a method comprising in vitro transcription of one or more DNA templates by an RNA polymerase. In embodiments, the in vitro-synthesized RNA encodes (or exhibits a coding sequence of) at least one protein or polypeptide. In some embodiments, the RNA encodes at least one protein that is capable of effecting a biological or biochemical effect when repeatedly or continuously introduced into a human or animal cell (e.g., a mammalian cell). In some embodiments, the disclosure comprises an RNA composition comprising or consisting of in vitro-synthesized RNA that encodes one protein or polypeptide. In embodiments, the disclosure comprises an RNA composition comprising or consisting of a mixture of multiple different in vitro-synthesized ssRNAs or mRNAs, each of which encodes a different protein. Other embodiments of the disclosure comprise an RNA composition comprising or consisting of in vitro-synthesized ssRNA that does not encode a protein or polypeptide, but instead exhibits the sequence of at least one long non-coding RNA (ncRNA). Still other embodiments comprise various reaction mixtures, kits and methods that comprise or use an RNA composition.

The terms “substantially free of dsRNA,” “virtually free of dsRNA,” “essentially free of dsRNA,” “practically free of dsRNA,” “extremely free of dsRNA,” or “absolutely free of dsRNA,” as used herein, respectively, that less than about: 0.5%, 0.1%, 0.05%, 0.01%, 0.001% or 0.0002% of the mass of the RNA in the treated ssRNA composition is dsRNA of a size greater than about 40 basepairs.

In embodiments, the one or more in vitro transcribed RNAs are substantially free of uncapped RNAs that exhibit a 5′-triphosphate group (which are considered to be one type of “contaminant RNA molecules” herein). As used herein, the RNA product is at least 50%, 60,%, 70%, 80%, 90%, 91%, 92%, 93%, 94%, 95%, 96%, 97%, 98%, 99%, 99.5%, or 99.99% free of uncapped RNAs that exhibit a 5′-triphosphate group.

As used herein, the term “quenching” refers to a process of deactivating any unreacted reagents.

Described herein are methods of producing transcribed RNA product with increased yield and reduced dsRNA impurities.

### EXAMPLES

### Example 1. IVT Preparation of mRNA

mRNA may be produced by a variety of in vitro transcription (IVT) methods. The following method, as well as the method in Example 2, were used in various experiments.
Step 1. 10× DNase I buffer (Table 1.1, item 3) was added to nuclease-free water (Table 1.1, item 2) and heated to 37±2° C. for 2 to 36 hours.

TABLE 1.1

| | | Amount | | |
|---|---|---|---|---|
| Item # | Description | (mL) | Stock Conc. | Final Conc. |
| 1 | IVT pool | 30.0 | 1X | 0.5X |
| 2 | Nuclease-free | 23.875 | n/a | n/a |
| | water | | | |
| 3 | 10X DNase I | 6.0 | 10X | 1X |
| | Buffer | | | |
| 4 | DNase I | 0.125 | 12.0 U/uL | 0.025 U/uL |

| | |
|---|---|
| Total Volume | 60.0 mL |

Step 2. 10×IVT buffer (400 mM Tris, 575 mM MgOAc, pH 7.6) was diluted 1:10 in nuclease-free water to yield 1×IVT buffer. A 50 mL reactor vessel was washed with 80 mL 1×IVT buffer. After IVT buffer wash, a sample was collected and the pH was recorded as pH 7.6.
Step 3. Meanwhile, 0.374 mL of 5M NaCl was added to 3.420 mL linearized DNA template “L. DNA” to obtain a total concentration of 62.3 mM NaCl, and the theoretical concentration of L. DNA was calculated to be 0.593 mg/mL.
Step 4. Nuclease-free water (Table 1.2, item 1) and 10×IVT buffer (Table 1.2, item 2) were added to the reactor from Step 2, and the reactor was warmed to 37±2° C.

TABLE 1.2

| | | Amount | | | | |
|---|---|---|---|---|---|---|
| Item # | Description | (mL) | Initial Conc | | Final Conc. | |
| 1 | Nuclease-free | 13.400 | n/a | n/a | n/a | n/a |
| | water | | | | | |
| 2 | 10X IVT buffer | 3.0 | 10 | X | 1 | X |

10X IVT buffer: 400 mM Tris, 575 mM MgOAc, pH 7.6

Step 5. Once the temperature of the reaction mixture reached 37±2° C., the following reagents (Table 1.3, items 3-10) were added to the reactor and stirred until the reaction temperature again reached 37±2° C.

TABLE 1.3

| Item | | Amount | | | | |
|---|---|---|---|---|---|---|
| # | Description | (mL) | Initial Conc. \| units | | Final Conc. \| units | |
| 3 | Ethanol | 1.50 | 100 | % | 5 | % |
| 4 | DTT | 0.450 | | M | 15 | mM |
| 5 | L. DNA | 3.794 | 0.593 | mg/mL | 0.075 | mg/mL |
| | template | | | | | |
| | from step 3 | | | | | |
| 6 | ATP | 1.062 | 325 | mM | 11.5 | mM |
| 7 | CTP | 1.367 | 325 | mM | 14.8 | mM |
| 8 | GTP | 1.385 | 325 | mM | 15.0 | mM |
| 9 | UTP | 0.637 | 325 | mM | 6.9 | mM |
| 10 | Capping | 0.450 | 100 | mM | 1.5 | mM |
| | reagent | | | | | |

Step 6. Once the temperature of the reaction mixture reached 37±2° C., the following reagents (Table 1.4, items 11-13) were added to the reactor and stirred at 37±2° C. for 150±3 min to generate the IVT pool.

TABLE 1.4

| Item # | Description | Amount (mL) | Initial Conc. \| units | | Final Conc. \| units | |
|---|---|---|---|---|---|---|
| 11 | RNase Inhibitor | 0.188 | 40 | U/uL | 0.25 | U/uL |
| 12 | IPPase | 0.060 | 1 | U/uL | 0.002 | U/uL |
| 13 | T7 RNA Polymerase | 2.709 | 2769 | U/uL | 250 | U/uL |

| | |
|---|---|
| Total Volume (Table 1.2-Table | 30.0 mL |
| 1.4, items 1-13; corresponds to | |
| Table 1.1, item 1) | |

Step 7. Between 140 and 150 min of IVT incubation, DNase I (Table 1.1, item 4) was added to the preheated DNase I buffer/water mixture of Step 1, to generate the DNase I mixture.
Step 8. Upon completion of IVT incubation (150 min), the DNase I mixture of Step 7 was added to the IVT pool from Step 6, and stirred at 37±2° C. for 15 to 20 minutes.
Step 9. The DNase reaction of Step 8 was quenched by adding 17.92 mL of 0.5 M EDTA (final concentration of 115 mM EDTA), the heating shut off, and the reaction was stirred for a minimum of 5 minutes to yield an IVT-DNase-EDTA pool volume of 77.92 mL. The quenched IVT-DNase-EDTA pool was aliquoted and stored at −70±10° C. immediately, or used directly.
Step 10. The product of Step 9 was optionally purified by silica column, and the concentration of silica-purified IVT crude was determined, e.g. by nanodrop.

### Example 2. IVT Preparation of mRNA

Step 1. 10× DNase I buffer (Table 2.1, item 3) was added to nuclease-free water (Table 2.1, item 2) and heated to 37±2° C. for 2 to 36 hours.

TABLE 2.1

| | | Amount | | |
|---|---|---|---|---|
| Item # | Description | (mL) | Stock Conc. | Final Conc. |
| 1 | IVT pool | 30.0 | 1X | 0.5X |
| 2 | Nuclease-free | 23.875 | n/a | n/a |
| | water | | | |
| 3 | 10X DNase I | 6.0 | 10X | 1X |
| | Buffer | | | |
| 4 | DNase I | 0.125 | 12.0 U/uL | 0.025 U/uL |

| | |
|---|---|
| Total Volume | 60.0 mL |

100 mM Tris, 25 mM MgCl₂, 5 mM CaCl₂, pH 7.6

Step 2. 10×IVT buffer (400 mM Tris, 575 mM MgOAc, pH 7.6) was diluted 1:10 in nuclease-free water to yield 1×IVT buffer. A 10 L reactor vessel was washed with 10 L 1×IVT buffer. After IVT buffer wash, a sample was collected and the pH was recorded as pH 7.45.
Step 3. Meanwhile, 49.4 mL of 5M NaCl was added to 225 mL of linearized DNA template “L. DNA” to obtain a total concentration of 0.9M NaCl, and the theoretical concentration of L. DNA was calculated to be 0.820 mg/mL. The volume of L.DNA and nuclease-free water needed for the reaction was calculated.
Step 4. Nuclease-free water (Table 2.2, item 1) and 10×IVT buffer (Table 2.2, item 2) were added to the reactor from Step 2, and the reactor was warmed to 37±2° C.
Step 5. Once the temperature of the reaction mixture reached 37±2° C., the following reagents (Table 2.2, items 3-10) were added to the reactor and stirred until the reaction temperature again reached

$${″}^{} \text{\textbackslash *} MERGEFORMAT \text{\textbackslash *} MERGEFORMAT 37 \pm 2 ° C .$$

Step 6. Once the temperature of the reaction mixture reached 37±2° C., reagents (Table 2.2, items 11-13) were added to the reactor and stirred at 37±2° C. for 105±5 min (or 150 min, or 200 min depending on the construct) to generate the IVT pool.

TABLE 2.2

| Item | | Amount | Amount | | | | |
|---|---|---|---|---|---|---|---|
| # | Description | (mL) | (g) | Initial Conc. \| units | | Final Conc. \| units | |
| 1 | Nuclease-free | 1619.706 | 1619.706 | | | | |
| | water | | | | | | |
| 2 | IVT buffer | 300 | 312 | 10 | X | 1 | X |
| 3 | 100% Ethanol | 150 | 119.4 | 100 | % | 5 | % |
| 4 | DTT | 45 | 46.8 | 1 | M | 15 | mM |
| 5 | L. DNA | 274.390 | 288.110 | 0.820 | mg/mL | 0.075 | mg/mL |
| | template from | | | | | | |
| | step 3 | | | | | | |
| 6 | ATP | 92 | 101.55 | 325 | mM | 10 | mM |
| 7 | CTP | 92 | 101.55 | 325 | mM | 10 | mM |
| 8 | GTP | 92 | 101.55 | 325 | mM | 10 | mM |
| 9 | UTP | 69.2 | 76.163 | 325 | mM | 7.5 | mM |
| 10 | Capping | 45 | 47.25 | 100 | mM | 1.5 | mM |
| | reagent | | | | | | |
| 11 | RNase | 18.750 | — | 40 | U/uL | 0.25 | U/uL |
| | Inhibitor | | | | | | |
| 12 | IPPase | 6 | — | 1 | U/uL | 0.002 | U/uL |
| 13 | T7 Polymerase | 225 | 261 | 1 | ug/uL | 0.075 | ug/uL |

10X IVT buffer: 400 mM Tris, 500 mM MgOAc, pH 7.6

Step 7. Between 90 to 105 min of IVT incubation, DNase I (Table 2.1, item 4) was added to the preheated DNase I buffer/water mixture of Step 1, to generate the DNase I mixture.
Step 8. Upon completion of IVT incubation, the DNase I mixture of Step 7 was added to the IVT pool from Step 6, and stirred at 37±2° C. for 15 to 20 minutes.
Step 9. The DNase reaction of Step 8 was quenched by adding 1500 mL (1659 mg) of 0.5 M EDTA (final concentration of 100 mM EDTA), the circulator setting was set to Tj=10° C., and the reaction was stirred for a minimum of 5 minutes to yield an IVT-DNase-EDTA pool volume of 7500 mL. The quenched IVT-DNase-EDTA pool was aliquoted and stored at −80° C. immediately, or held at 2° C. to 8° C. and used directly.
Step 10. The product of Step 9 was optionally purified by silica column, and the concentration of silica-purified IVT crude was determined, e.g. by nanodrop.

### Example 3. DT Purification of DNAse Treated and EDTA Quenched IVT of mRNA Diluted with Water for Injection (WFI)

A DNAse treated, EDTA quenched mRNA referred to as RNA #1 was prepared by IVT as described in Example 1. After silica column purification (Example 1, step 10), the conductivity was 21.78 mS/cm and the purity was 68%. RNA #1 was diluted with water for injection (WFI) as provided in Table 3.2 below. The conductivity was determined, and then each sample was purified on a 4 mL Oligo dT column (Item No.: BIA-904.1219-2 from Bia Separations, which has a Poly dT Ligand 18 nucleotides in length attached via a C12 linker). The conductivity of WFI=4.579 uS/cm measured at ambient temperature.

Purifications were performed in accordance with the method provided in Table 3.1. The instrument used for monitoring and loading was an AKTA Avant 150 using a Teledyne Autosampler. Samples were collected in 50 mL collection tubes and had their volume measured by weighing the sample and the concentration assessed by nanodrop spectrophotometer. Samples were normalized to 100 ng/μl and loaded on the fragment analyzer instrument for purity assessment.

TABLE 3.1

| Step # | Column step | Buffer | CVs | Day |
|---|---|---|---|---|
| 1 | Equilibration | 50% dT low-salt | 5 | Repeat for |
| | | 50% dT high-salt | | each cycle |
| 2 | Load | 50% IVT-DNase- | Varies Based | |
| | | EDTA-Water | on Dilution | |
| | | 50% dT high-salt | | |
| 3 | Wash 1 | 50% dT low-salt | 3 | |
| | | 50% dT high-salt | | |
| 4 | Wash 2 | 2.5M Guanidine | 5 | |
| | | HCl, 100 mM | | |
| | | Tris pH 8.0 | | |
| 5 | Wash 3 | 100% dT High Salt | 5 | |
| 6 | Wash 4 | dT Low Salt | 7 | |
| 7 | Elute | WFI | 1.5 + hold | |
| 8 | Strip | WFI | 3 | |
| 9 | CIP | 0.1M NaOH | 5 | Repeat for |
| | | | | each cycle |
| 10 | Neutralization | dT Low Salt | 5 | Repeat for |
| | | | | each cycle |

dT Low Salt = 50 mM Sodium Phosphate, 2 mM EDTA, pH 7.0;
dT High Salt = 1000 mM Sodium Chloride, 50 mM Sodium Phosphate, 2 mM EDTA, pH 7.0

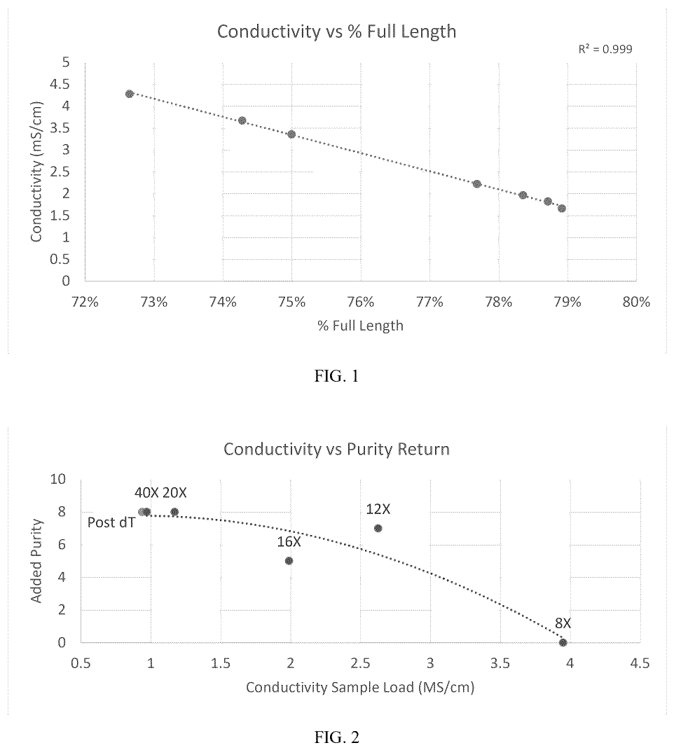

RNA purity (% full length) was estimated by fragment P+H. P+H=integrated P (primary peak)+H (high molecular weight impurities), excluding L (low molecular weight impurities), wherein P+H+L=100%. See Table 3.2. A clear correlation was identified between conductivity prior to dT column, and % full length, as shown in FIG. 1.

TABLE 3.2

| IVT Loading | | | | | | | Fragment |
|---|---|---|---|---|---|---|---|
| Fold | Resin Challenge | Cond. Post | dT Recovery | | | | Avg P + H |
| Dilution | (mg/mL)* | Dilution (mS/cm) | V | [c] | Y | % Rec | Purity |
| 7 | 2 | 4.28 | 14.8 | 488.7 | 7.23 | 90%*** | 73% |
| 9 | 2 | 3.671 | 8 | 821.7 | 6.57 | 82% | 74% |
| 11 | 2 | 3.357 | 7.84 | 860.7 | 6.75 | 84% | 75% |
| 15 | 2 | 2.223 | 9.08 | 678.4 | 6.16 | 77% | 78% |
| 17 | 2 | 1.969 | 7.9 | 832.3 | 6.58 | 82% | 78% |
| 19 | 2 | 1.824 | 7.72 | 832.9 | 6.43 | 80% | 79% |
| 21 | 2 | 1.664 | 7.74 | 831.6 | 6.44 | 80% | 79% |

Resin challenge = mass of RNA loaded on a column, divided by the volume of the column. V = amount recovered (grams). [c] = concentration of sample recovered in ng/uL, as measured by nanodrop spectrophotometer. Y = yield (mg). % Rec = percentage recovered based on the theoretical resin challenge.
*Resin challenge was based on theoretical 12 g/L IVT. Using a 2 mg/mL resin challenge, a 4 mL Oligo dT column was loaded with 8 mg RNA (0.667 mL of the 12 g/L IVT). Given that the dilution was different for each condition, the column was loaded with larger volumes for larger dilutions.
***Sample load was slightly higher than the other samples, due to having the sample line pre-filled; therefore, the calculated % Rec was likely higher than actual recovery.

### Example 4. DT Purification of DNAse Treated and EDTA Quenched IVT of mRNA Diluted with Water for Injection (WFI)

A DNAse treated, EDTA quenched mRNA referred to as RNA #2 was prepared by IVT as in Example 2 and diluted with buffer or with water for injection (WFI) as provided in Table 4.2 below. The conductivity was determined, and then each sample was purified on an 8 mL Oligo dT column (from Bia Separations, which has a Poly dT Ligand 18 nucleotides in length attached via a C6 or C12 linker). Purifications were performed in accordance with the method provided in Table 4.1. The instrument used for monitoring and loading was an AKTA Avant 150 using a Teledyne Autosampler. Samples were collected in 50 mL collection tubes and had their volume measured by weighing the sample and the concentration assessed by nanodrop spectrophotometer. Samples were normalized to 100 ng/ul and loaded on the fragment analyzer instrument for purity assessment.

TABLE 4.1

| Step # | Column step | Buffer | CVs | Day |
|---|---|---|---|---|
| 1 | Equilibration | 10% Buffer C | 5 | Repeat for |
| | | 90% dT low salt | | each cycle |
| 2 | Load | 50% IVT-DNase- | Varies Based | |
| | | EDTA-Water | on Dilution | |
| | | 50% dT high-salt | | |
| 3 | Wash 1 | 10% Buffer C | 5 | |
| | | 90% dT low salt | | |
| 4 | Wash 2 | dT Low Salt | 7 | |
| 5 | Elute | WFI | 1.5 + hold | |
| 6 | Strip | WFI | 3 | |
| 7 | CIP | 0.1M NaOH | 5 | Repeat for |
| | | | | each cycle |
| 8 | Neutralization | dT Low Salt | 5 | Repeat for |
| | | | | each cycle |

dT Low Salt = 50 mM Sodium Phosphate, 2 mM EDTA, pH 7.0;
dT High Salt = 1000 mM Sodium Chloride, 50 mM Sodium Phosphate, 2 mM EDTA, pH 7.0
Buffer C = 5M NaCl, 50 mM Sodium Phosphate, 2 mM EDTA

A clear improvement in purity was achieved when IVT was diluted in water, as shown in Table 4.2 below and FIG. 2. In addition, re-purification of the elution pool from the STD condition (which exhibited a conductivity of 0.9379 mS/cm) under the same STD conditions resulted in a +8% purity return (see FIG. 2, “Post dT” datapoint), similar to the purity return for a single purification of the 20× and 40× dilutions with water. This demonstrated that the benefit of a second dT purification was not due to the use of a second chromatography step, but instead was the result of the mRNA's dilution to a lower conductivity threshold. Indeed, the mRNA recovery and throughput was improved by diluting the mRNA, as compared to running two sequential oligo dT purifications.

TABLE 4.2

| | | | Purity | Purity |
|---|---|---|---|---|
| | Sample Load | Conductivity | Return | Return |
| Condition | (Preload Dilution) | mS/cm | (C6) | (C12) |
| STD | 8X with Buffer A | 8.425 | 77% | 76% |
| 1 | 8X with Water | 3.949 | +2% | +0% |
| 3 | 12X with Water | 2.626 | | +7% |
| 4 | 16X with Water | 1.988 | | +5% |
| 5 | 20X with Water | 1.170 | +4% | +8% |
| 6 | 40X with Water | 0.9713 | +4% | +8% |

Buffer A: 50 mM Sodium Phosphate, 2 mM EDTA

### Example 5. DT Purification of DNAse Treated and EDTA Quenched IVT of mRNA Diluted with Water for Injection (WFI)

A DNAse treated, EDTA quenched mRNA referred to as RNA #2 was prepared by IVT as in Example 1 and diluted with water for injection (WFI) as indicated in Table 7.1 below. The conductivity was determined, and then each sample was purified on a 4 mL Oligo dT column (Item No.: BIA-904.1219-2 from Bia Separations, which has a Poly dT Ligand 18 nucleotides in length attached via a C12 linker) in accordance with the method provided in Table 5.1. The instrument used for monitoring and loading was an AKTA Avant 150 using a Teledyne Autosampler. Samples were collected in 50 mL collection tubes and had their volume measured by weighing the sample and the concentration assessed by nanodrop spectrophotometer. Samples were normalized to 100 ng/ul and loaded on the fragment analyzer instrument for purity assessment.

TABLE 5.1

| Step # | Column step | Buffer | CVs | Day |
|---|---|---|---|---|
| 1 | Equilibration | 50% dT low-salt | 5 | Repeat for |
| | | 50% dT high-salt | | each cycle |
| 2 | Load | 50% IVT-DNase- | Varies Based | |
| | | EDTA- Water | on Dilution | |
| | | 50% dT high-salt | | |
| 3 | Wash 1 | 50% dT low-salt | 5 | |
| | | 50% dT high-salt | | |
| 4 | Wash 2 | dT Low Salt | 7 | |
| 5 | Elute | WFI | 1.5 + hold | |
| 6 | Strip | WFI | 3 | |
| 7 | CIP | 0.1M NaOH | 5 | Repeat for |
| | | | | each cycle |
| 8 | Neutralization | dT Low Salt | 5 | Repeat for |
| | | | | each cycle |

dT Low Salt = 50 mM Sodium Phosphate, 2 mM EDTA, pH 7.0;
dT High Salt = 1000 mM Sodium Chloride, 50 mM Sodium Phosphate, 2 mM EDTA, pH 7.0

TABLE 5.2

| | | | | | Load | Recovery | % |
|---|---|---|---|---|---|---|---|
| Sample | L | P | H | P + H | (mg) | (mg) | Yield |
| B2 (16 mg, STD 8X Buffer A dilution) | 27 | 71 | 2 | 73 | 16 | 16 | 98 |
| B3 (24 mg, 20X water dilution, | 20 | 78 | 3 | 80 | 24 | 14 | 60 |
| cycle 1) | | | | | | | |
| B4 (24 mg, 20X water dilution, | 19 | 79 | 3 | 81 | 24 | 15 | 65 |
| cycle 2) | | | | | | | |
| B5 (32 mg, 20X water dilution, | 26 | 72 | 3 | 74 | 32 | 16 | 52 |
| cycle 1) | | | | | | | |
| B6 (32 mg, 20X water dilution, | 20 | 78 | 3 | 81 | 32 | 16 | 50 |
| cycle 2) | | | | | | | |
| B7 (8 mg, 12X water dilution) | 20 | 77 | 3 | 80 | 8 | 5 | 65 |
| B8 (16 mg, 12X water dilution) | 21 | 79 | 3 | 80 | 16 | 11 | 70 |
| B9 (24 mg, 12X water dilution) | 19 | 79 | 3 | 81 | 24 | 15 | 61 |
| B10 (20 mg, 12X water dilution, | 19 | 79 | 2 | 81 | 20 | 13 | 66 |
| cycle 1) | | | | | | | |
| B11(20 mg, 12X water dilution, | 18 | 79 | 4 | 82 | 20 | 13 | 65 |
| cycle 2) | | | | | | | |
| B12 (8 mg, 16X water dilution) | 19 | 78 | 3 | 81 | 8 | 5 | 62 |
| C1 (16 mg, 16X water dilution) | 23 | 74 | 4 | 78 | 16 | 11 | 68 |
| C2 (24 mg, 16X water dilution) | 19 | 78 | 2 | 81 | 24 | 14 | 61 |
| C3 (20 mg, 16X water dilution, | 20 | 77 | 3 | 80 | 20 | 13 | 65 |
| cycle 1) | | | | | | | |
| C4 (20 mg, 16X water dilution, | 18 | 79 | 3 | 82 | 20 | 13 | 64 |
| cycle 2) | | | | | | | |

Buffer A: 50 mM Sodium Phosphate, 2 mM EDTA

### Example 6. DT Purification of DNAse Treated and EDTA Quenched IVT of mRNA Diluted with Alternative Low Conductivity Diluents

A DNAse treated, EDTA quenched mRNA referred to as RNA #3 was prepared by IVT as Tables 6.0.1-6.0.3 below, and diluted as indicated in Table 6.2 below. The conductivity was determined, and then each sample was purified on a 4 mL Oligo dT column (Item No.: BIA-904.1219-2 from Bia Separations, which has a Poly dT Ligand 18 nucleotides in length attached via a C12 linker) in accordance with the method provided in Table 6.1 below. The instrument used for monitoring and loading was an AKTA Avant 150 using a Teledyne Autosampler. Samples were collected in 50 mL collection tubes and had their volume measured by weighing the sample and the concentration assessed by nanodrop spectrophotometer. Samples were normalized to 100 ng/μl and loaded on the fragment analyzer instrument for purity assessment. As indicated in Table 6.2, all 15× diluted “low-conductivity” conditions resulted in purity improvements relative to dilutions in dT low salt buffer. 15× dilution in 20% ethanol also achieved a purity improvement (data not shown).

TABLE 6.01

| Item # | Description | Amount (uL) | Initial Conc. \| units | | Final Conc. \| units | |
|---|---|---|---|---|---|---|
| 1 | Nuclease-free water | 4434.13 | | | | |
| 2 | Tris, pH 7.5 | 320 | 1000 | mM | 40 | mM |
| 3 | MgOAc | 400 | 1000 | mM | 50 | mM |
| 4 | DTT | 120 | 1000 | mM | 15 | mM |
| 5 | EtOH | 400 | 100 | % | 5 | % |
| 6 | L. DNA | 699.68 | 857.53 | ng/uL | 75 | ng/uL |
| 7 | ATP | 295.38 | 325 | mM | 12 | mM |
| 8 | CTP | 295.38 | 325 | mM | 12 | mM |
| 9 | GTP | 295.38 | 325 | mM | 12 | mM |
| 10 | UTP | 147.69 | 325 | mM | 6 | mM |
| 11 | Capping Reagent | 120 | 100 | mM | 1.5 | mM |
| 12 | RNase Inhibitor | 50 | 40 | U/uL | 0.25 | U/uL |
| 13 | IPPase | 16 | 1 | U/uL | 0.002 | U/uL |
| 14 | T7 Polymerase | 406.34 | 2461 | U/uL | 125 | U/uL |
| | Total IVT | 8000 | | | | |

TABLE 6.0.2

| | Amount | Initial | | Final | |
|---|---|---|---|---|---|
| Description | (μL) | Conc. \| units | | Conc. \| units | |
| Nuclease-free water | 6366.56 | | | | |
| 10X DNAse buffer | 1600.00 | 10 | X | 1 | X |
| DNAse I | 33.54 | 11.926 | U/uL | 0.025 | U/uL |
| DNAse + IVT total | 16000 | | | | |

TABLE 6.0.3

| | | | |
|---|---|---|---|
| | Amount | Initial | Final |
| Description | (μL) | Conc. \| units | Conc. \| units |

| | | | | | |
|---|---|---|---|---|---|
| EDTA | 4000 | 0.5 | M | 100 | mM |
| DNAse + | 20000 | | | | |
| IVT + _EDTA | | | | | |

TABLE 6.1

| Step # | Column step | Buffer | CVs | Day |
|---|---|---|---|---|
| 1 | Equilibration | 50% dT low-salt | 5 | Repeat for |
| | | 50% dT high-salt | | each cycle |
| 2 | Load | 50% IVT-DNase- | Varies Based | |
| | | EDTA-Diluent | on Dilution | |
| | | (e.g. in water) | | |
| | | 50% dT high-salt | | |
| 3 | Wash 1 | 50% dT low-salt | 5 | |
| | | 50% dT high-salt | | |
| 4 | Wash 2 | dT Low Salt | 7 | |
| 5 | Elute | WFI | 1.5 + hold | |
| 6 | Strip | WFI | 3 | |
| 7 | CIP | 0.1M NaOH | 5 | Repeat for |
| | | | | each cycle |
| 8 | Neutralization | dT Low Salt | 5 | Repeat for |
| | | | | each cycle |

dT Low Salt = 50 mM Sodium Phosphate, 2 mM EDTA, pH 7.0;
dT High Salt = 1000 mM Sodium Chloride, 50 mM Sodium Phosphate, 2 mM EDTA, pH 7.0

TABLE 6.2

| | Conductivity | |
|---|---|---|
| Sample | mS/cm | P + H |
| B3: 10X dilution in dT Low Salt (Control) | 5.2649 | 75% |
| B4: 15X dilution in water | 1.7427 | 79% |
| B5: 15X dilution in 20% DMSO (final | 0.4646* | 80% |
| concentration of 18.7% DMSO) | | |
| B6: 15X dilution in 1M urea (final | 1.4602 | 81% |
| concentration of 0.93M urea) | | |

dT Low Salt = 50 mM Sodium Phosphate, 2 mM EDTA, pH 7.0
*conductivity in organic solutions is not able to be measured accurately

### Example 7. DT Purification of In Vitro Transcription Product with Urea Dilution

Frozen IVT products at 4 g/L were thawed and diluted with a number of diluents containing urea (as shown in Table 7.1). Dilution ratios were selected based on previous experiments such as Example 4 to attempt to optimise purity increased and processing time. The resulting diluted samples were assessed for conductivity. The diluted samples were purified on a 4 mL Oligo dT column (Item No.: BIA-904.1219-2 from Bia Separations, which has a Poly dT Ligand 18 nucleotides in length attached via a C12 linker) in accordance with the method provided in Table 6.1 in Example 6 with modifications as described in this Example. For example, the nature of the diluent used is as specified in this Example. The instrument used for monitoring and loading was an AKTA Avant 150 using a Teledyne Autosampler. Samples were collected in 50 mL collection tubes and had their volume measured by weighing the sample and the concentration assessed by nanodrop spectrophotometer. Samples were normalized to 100 ng/μl and loaded on the fragment analyzer instrument for purity assessment. The purity results in terms of L, P and H are shown in Table 7.1.

TABLE 7.1
Diluents Containing Urea and Purification Conditions and Purity Results

| | | | Sample | | | | |
|---|---|---|---|---|---|---|---|
| | | | Conc. at | | | | |
| | Dilution | Loading | loading | | | | |
| No | Condition | condition | (g/L) | Notes | L | P | H |
| Ctrl | IVT Crude | / | 4 g/L | Control | 24% | 76% | 0% |
| | (control) | | undiluted | | | | |
| 1 | 1:1 | 1:1 inline | 1 | 1.5 g/L resin | 17% | 82% | 0% |
| | with 6M Urea | with dT High Salt | | challenge | | | |
| 2 | 1:1 | 1:1 inline | 1 | Equilibration | 16% | 83% | 1% |
| | with 6M Urea | with dT High Salt | | and Wash 1 with | | | |
| | | | | 6M urea | | | |
| 3 | 1:1 | 1:10 inline | 0.2 | 1.5 g/L resin | 16% | 83% | 1% |
| | with 6M Urea | with dT High Salt | | challenge | | | |
| 4 | 1:15 | 1:1 inline | 0.133 | 1.5 g/L resin | 12% | 87% | 1% |
| | with 6M Urea | with dT High Salt | | challenge | | | |
| 5 | 1:15 | 1:10 inline | 0.0266 | 1.5 g/L resin | 12% | 87% | 0% |
| | with 6M Urea | with dT High Salt | | challenge | | | |
| 6 | 1:15 | 1:1 inline | 0.133 | 1.5 g/L resin | 14% | 85% | 1% |
| | with water | with dT High Salt | | challenge | | | |
| | (WFI) | | | | | | |
| 7 | 1:15 | 1:1 inline | 0.0266 | 1.5 g/L resin | 11% | 85% | 4% |
| | with water | with dT High Salt | | challenge | | | |
| | (WFI) | | | | | | |

6M Urea = urea in water such as WFI or ultrapure water
dT High Salt = 1000 mM Sodium Chloride, 50 mM Sodium Phosphate, 2 mM EDTA, pH 7.0
L: Low molecular weight impurities; P: Primary peak; H: High molecular weight impurities.
L + P + H = 100%

Dilution with more urea (e.g. 1:15 urea) exhibited an improvement similar to higher water dilution (1:15 WFI). Nevertheless, higher urea dilution showed highest purity (in terms of P).

Residual DNA amount was measured and the results shown in Table 7.2.

TABLE 7.2
Residual DNA

| | Dilution | Loading | | Residual DNA |
|---|---|---|---|---|
| No | Condition | condition | Notes | pg/mg |
| 1 | 1:1 with | 1:1 inline with | 1.5 g/L resin | 1212 |
| | 6M Urea | dT High Salt | challenge | |
| 2 | 1:1 with | 1:1 inline with | Equilibration | 1608 |
| | 6M Urea | dT High Salt | and Wash 1 | |
| | | | with 6M urea | |
| 3 | 1:1 with | 1:10 inline with | 1.5 g/L resin | 1405 |
| | 6M Urea | dT High Salt | challenge | |
| 4 | 1:15 with | 1:1 inline with | 1.5 g/L resin | 385 |
| | 6M Urea | dT High Salt | challenge | |
| 5 | 1:15 with | 1:10 inline with | 1.5 g/L resin | 435 |
| | 6M Urea | dT High Salt | challenge | |
| 6 | 1:15 with | 1:1 inline with | 1.5 g/L resin | 1263 |
| | water (WFI) | dT High Salt | challenge | |
| 7 | 1:15 with | 1:1 inline with | 1.5 g/L resin | 1440 |
| | water (WFI) | dT High Salt | challenge | |

6M Urea = urea in water such as WFI or ultrapure water

Higher dilution at 1:15 with urea exhibited the lowest residual DNA. Both low dilution ratio and dilution with water at high dilution ratio showed high amount of residual DNA.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1.-75. (canceled)

76. A method of purifying a nucleic acid comprising an RNA transcription product, the method comprising:
(a) providing an RNA transcription product composition comprising the RNA transcription product and having a conductivity of less than 20 mS/cm;
(b) introducing the RNA transcription product composition to a polynucleotide affinity column; and
(c) eluting the RNA transcription product from the polynucleotide affinity column to obtain a purified RNA transcription product.

77. The method of claim 76, wherein providing the RNA transcription product composition of step (a) comprises obtaining a crude RNA transcript composition from an in vitro transcription reaction and adjusting the conductivity of the crude RNA transcript composition to be less than 20 mS/cm.

78. The method of claim 76, wherein adjusting the conductivity comprises diluting the crude RNA transcript composition with a conductivity decreasing agent.

79. The method of claim 76, wherein the conductivity of the RNA transcription product composition is less than 19 mS/cm, less than 18 mS/cm, less than 17 mS/cm, less than 16 mS/cm, less than 15 mS/cm, less than 14 mS/cm, less than 13 mS/cm, less than 12 mS/cm, less than 11 mS/cm, less than 10 mS/cm, less than 9 mS/cm, less than 8 mS/cm, less than 7 mS/cm, less than 6 mS/cm, less than 5 mS/cm, less than 4 mS/cm, less than 3 mS/cm, less than 2 mS/cm, or less than 1 mS/cm.

80. The method of claim 76, wherein
(i) the purified RNA transcription product has a purity of at least 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9%; or
(ii) the method produces eluted, purified, or enriched nucleic acid which is at least 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% free of contaminants.

81. The method of claim 76, wherein
(i) providing the RNA transcription product composition of step (a) comprises obtaining a crude RNA transcript composition from an in vitro transcription reaction and adjusting the conductivity of the crude RNA transcript composition to 5 mS/cm or lower; and wherein the purified RNA transcription product has a purity of at least 73%; or
(ii) providing the RNA transcription product composition of step (a) comprises obtaining a crude RNA transcript composition from an in vitro transcription reaction and adjusting the conductivity of the crude RNA transcript composition to be 4 mS/cm or lower; and wherein the purified RNA transcription product has a purity of at least 75%.

82. The method of claim 76, wherein the RNA transcription product composition is from an in vitro transcription reaction.

83. The method of claim 76, wherein the RNA transcription product composition comprises a DNase, ethylenediaminetetraacetic acid (EDTA), free nucleotides, a buffer, an RNA, or any combination of the foregoing.

84. The method of claim 83, wherein
(i) the RNA comprises a self-amplifying RNA; or
(ii) the RNA comprises an mRNA; or
(iii) the RNA comprises a poly-A tail.

85. The method of claim 77, wherein
(i) adjusting the conductivity of the composition comprises diluting the composition; or
(ii) adjusting the conductivity of the composition comprises diluting the composition with a conductivity decreasing agent; or
(iii) adjusting the conductivity of the composition comprises diluting the composition by a factor of about 1.125×, about 1.25×, about 1.5×, about 1.75×, about 2×, about 2.5×, about 3×, about 4×, about 5×, about 6×, about 7×, about 8×, about 9×, about 10×, about 11×, about 12×, about 13×, about 14×, or about 15×, or a range defined by any two of the aforementioned factors; or
(iv) adjusting the conductivity of the composition comprises diluting the composition with a conductivity decreasing agent by a factor of about 1.125×, about 1.25×, about 1.5×, about 1.75×, about 2×, about 2.5×, about 3×, about 4×, about 5×, about 6×, about 7×, about 8×, about 9×, about 10×, about 11×, about 12×, about 13×, about 14×, or about 15×, or a range defined by any two of the aforementioned factors.

86. The method of claim 85, wherein
(i) the conductivity decreasing agent comprises water, ethanol, dimethylsulfoxide, urea, or a combination thereof; or
(ii) the conductivity decreasing agent comprises a low-conductivity solution; or
(iii) the conductivity decreasing agent is sterile; or
(iv) the conductivity decreasing agent is formulated for an injection; or
(v) the conductivity decreasing agent comprises urea; or
(vi) the conductivity decreasing agent is about 0.5M to about 10M, about 1M to about 9M, about 3M to about 8M, about 4M to about 7M, about 5M to about 7M, about 5.5M to about 6.5M, or about 6M aqueous urea; or
(vii) the conductivity decreasing agent is water.

87. The method of claim 85, wherein the conductivity decreasing agent has a conductivity of less than 20 mS/cm, less than 19 mS/cm, less than 18 mS/cm, less than 17 mS/cm, less than 16 mS/cm, less than 15 mS/cm, less than 14 mS/cm, less than 13 mS/cm, less than 12 mS/cm, less than 11 mS/cm, less than 10 mS/cm, less than 9 mS/cm, less than 8 mS/cm, less than 7 mS/cm, less than 6 mS/cm, less than 5 mS/cm, less than 4 mS/cm, less than 3 mS/cm, less than 2 mS/cm, or less than 1 mS/cm.

88. The method of claim 85, wherein the conductivity decreasing agent has a conductivity of less than 1 mS/cm or less than 10 μS/cm.

89. The method of claim 76, wherein
(i) the polynucleotide affinity column comprises an oligo dT column; or
(ii) the polynucleotide affinity column comprises a solid support; or
(iii) the polynucleotide affinity column comprises a solid support comprising a matrix, gel, silica, or bead.

90. The method of claim 76, wherein introducing the composition to the polynucleotide affinity column comprises binding the nucleic acid to a component within the column.

91. The method of claim 76, further comprising one or more of the following:
(i) performing a wash on the polynucleotide affinity column while the nucleic acid is bound to a component within the column;
(ii) eluting the nucleic acid from the polynucleotide affinity column;
(iii) performing an additional enrichment or purification process on the composition or nucleic acid after introducing the composition to the polynucleotide affinity column, or after eluting the nucleic acid from the polynucleotide affinity column;
(iv) performing an additional enrichment or purification process on the composition or nucleic acid after introducing the composition to the polynucleotide affinity column, or after eluting the nucleic acid from the polynucleotide affinity column, wherein the additional enrichment or purification process comprises chromatography; and
(v) performing an additional enrichment or purification process on the composition or nucleic acid after introducing the composition to the polynucleotide affinity column, or after eluting the nucleic acid from the polynucleotide affinity column, wherein the additional enrichment or purification process comprises affinity chromatography, high-performance liquid chromatography, silica purification, or ultrafiltration and diafiltration (UFDF).

92. A method of enriching or purifying a nucleic acid, the method comprising:
obtaining a conductivity measurement of a composition comprising the nucleic acid; and
when the conductivity measurement is at or below a threshold, enriching or purifying the nucleic acid from the composition.

93. The method of claim 92, further comprising:
when the conductivity measurement is not at or below a threshold, adjusting the conductivity of the composition to be at or below the threshold before enriching or purifying the nucleic acid from the composition.

94. The method of claim 92, further comprising providing or obtaining the composition comprising the nucleic acid by performing in vitro transcription.

95. The method of claim 92, wherein
(a) the nucleic acid comprises a transcription product; or
(b) the nucleic acid comprises an RNA; or
(c) the nucleic acid comprises a self-amplifying RNA; or
(d) the nucleic acid comprises an mRNA; or
(e) the nucleic acid comprises an RNA having a poly-A tail; or
(f) the composition comprising the nucleic acid is an RNA transcription product composition; or
(g) the composition comprising the nucleic acid is an RNA transcription product composition from an in vitro transcription reaction.

96. The method of claim 92, wherein obtaining the conductivity measurement comprises determining an ability of the composition to conduct electricity.

97. The method of claim 92, wherein
(a) the threshold is less than 30 mS/cm, less than 29 mS/cm, less than 28 mS/cm, less than 27 mS/cm, less than 26 mS/cm, less than 25 mS/cm, less than 24 mS/cm, less than 23 mS/cm, less than 22 mS/cm, less than 21 mS/cm, less than 20 mS/cm, less than 19 mS/cm, less than 18 mS/cm, less than 17 mS/cm, less than 16 mS/cm, less than 15 mS/cm, less than 14 mS/cm, less than 13 mS/cm, less than 12 mS/cm, less than 11 mS/cm, less than 10 mS/cm, less than 9 mS/cm, less than 8 mS/cm, less than 7 mS/cm, less than 6 mS/cm, less than 5 mS/cm, less than 4 mS/cm, less than 3 mS/cm, less than 2 mS/cm, or less than 1 mS/cm; or
(b) the threshold is about 30 mS/cm, about 29 mS/cm, about 28 mS/cm, about 27 mS/cm, about 26 mS/cm, about 25 mS/cm, about 24 mS/cm, about 23 mS/cm, about 22 mS/cm, about 21 mS/cm, about 20 mS/cm, about 19 mS/cm, about 18 mS/cm, about 17 mS/cm, about 16 mS/cm, about 15 mS/cm, about 14 mS/cm, about 13 mS/cm, about 12 mS/cm, about 11 mS/cm, about 10 mS/cm, about 9 mS/cm, about 8 mS/cm, about 7 mS/cm, about 6 mS/cm, about 5 mS/cm, about 4 mS/cm, about 3 mS/cm, about 2 mS/cm, or about 1 mS/cm; or
(c) the threshold is 30 mS/cm, 29 mS/cm, 28 mS/cm, 27 mS/cm, 26 mS/cm, 25 mS/cm, 24 mS/cm, 23 mS/cm, 22 mS/cm, 21 mS/cm, 20 mS/cm, 19 mS/cm, 18 mS/cm, 17 mS/cm, 16 mS/cm, 15 mS/cm, 14 mS/cm, 13 mS/cm, 12 mS/cm, 11 mS/cm, 10 mS/cm, 9 mS/cm, 8 mS/cm, 7 mS/cm, 6 mS/cm, 5 mS/cm, 4 mS/cm, 3 mS/cm, 2 mS/cm, or 1 mS/cm.

98. The method of claim 93, wherein
(a) adjusting the conductivity of the composition comprises diluting the composition; or
(b) adjusting the conductivity of the composition comprises diluting the composition with a conductivity decreasing agent; or
(c) adjusting the conductivity of the composition comprises diluting the composition by a factor of about 1.125×, about 1.25×, about 1.5×, about 1.75×, about 2×, about 2.5×, about 3×, about 4×, about 5×, about 6×, about 7×, about 8×, about 9×, about 10×, about 11×, about 12×, about 13×, about 14×, or about 15×, or a range defined by any two of the aforementioned factors; or
(d) adjusting the conductivity of the composition comprises diluting the composition with a conductivity decreasing agent by a factor of about 1.125×, about 1.25×, about 1.5×, about 1.75×, about 2×, about 2.5×, about 3×, about 4×, about 5×, about 6×, about 7×, about 8×, about 9×, about 10×, about 11×, about 12×, about 13×, about 14×, or about 15×, or a range defined by any two of the aforementioned factors.

99. The method of claim 98, wherein
(i) the conductivity decreasing agent comprises water, ethanol, dimethylsulfoxide, urea, or a combination thereof; or
(ii) the conductivity decreasing agent comprises a low-conductivity solution; or
(iii) the conductivity decreasing agent is sterile; or
(iv) the conductivity decreasing agent is formulated for an injection; or
(v) the conductivity decreasing agent comprises urea; or
(vi) the conductivity decreasing agent is about 0.5M to about 10M, about 1M to about 9M, about 3M to about 8M, about 4M to about 7M, about 5M to about 7M, about 5.5M to about 6.5M, or about 6M aqueous urea; or
(vii) the conductivity decreasing agent is water.

100. The method of claim 98, wherein the conductivity decreasing agent has a conductivity of less than 20 mS/cm, less than 19 mS/cm, less than 18 mS/cm, less than 17 mS/cm, less than 16 mS/cm, less than 15 mS/cm, less than 14 mS/cm, less than 13 mS/cm, less than 12 mS/cm, less than 11 mS/cm, less than 10 mS/cm, less than 9 mS/cm, less than 8 mS/cm, less than 7 mS/cm, less than 6 mS/cm, less than 5 mS/cm, less than 4 mS/cm, less than 3 mS/cm, less than 2 mS/cm, or less than 1 mS/cm.

101. The method of claim 98, wherein the conductivity decreasing agent has a conductivity of less than 1 mS/cm or less than 10 μS/cm.

102. The method of claim 92, wherein
(a) enriching or purifying the nucleic acid from the composition comprises performing affinity chromatography on the composition; or
(b) enriching or purifying the nucleic acid from the composition comprises performing affinity chromatography on the composition comprising introducing the composition to a polynucleotide affinity column; or
(c) enriching or purifying the nucleic acid from the composition comprises performing affinity chromatography on the composition comprising introducing the composition to an oligo dT polynucleotide affinity column; or
(d) enriching or purifying the nucleic acid from the composition comprises performing affinity chromatography on the composition comprising introducing the composition to a polynucleotide affinity column, wherein the polynucleotide affinity column comprises a solid support; or
(e) enriching or purifying the nucleic acid from the composition comprises performing affinity chromatography on the composition comprising introducing the composition to a polynucleotide affinity column, wherein the polynucleotide affinity column comprises a solid support comprising a matrix, gel, silica, or bead; or
(f) enriching or purifying the nucleic acid from the composition comprises performing affinity chromatography on the composition comprising introducing the composition to a polynucleotide affinity column; wherein introducing the composition to the polynucleotide affinity column comprises binding the nucleic acid to a component within the column.

103. The method of claim 102, further comprising one or more of the following:
(A) performing a wash on the polynucleotide affinity column while the nucleic acid is bound to a component within the column;
(B) eluting the nucleic acid from the polynucleotide affinity column;
(C) performing an additional enrichment or purification process on the composition or nucleic acid after introducing the composition to the polynucleotide affinity column, or after eluting the nucleic acid from the polynucleotide affinity column;
(D) performing an additional enrichment or purification process on the composition or nucleic acid after introducing the composition to the polynucleotide affinity column, or after eluting the nucleic acid from the polynucleotide affinity column, wherein the additional enrichment or purification process comprises chromatography; and
(E) performing an additional enrichment or purification process on the composition or nucleic acid after introducing the composition to the polynucleotide affinity column, or after eluting the nucleic acid from the polynucleotide affinity column, wherein the additional enrichment or purification process comprises affinity chromatography, high-performance liquid chromatography, silica purification, or ultrafiltration and diafiltration (UFDF).

104. The method of claim 92, wherein the method produces eluted, purified, or enriched nucleic acid which is at least 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% free of contaminants.

105. The method of claim 92, wherein
(a) prior to obtaining the conductivity measurement, the composition or nucleic acid has undergone an additional enrichment or purification process; or
(b) prior to obtaining the conductivity measurement, the composition or nucleic acid has not undergone an additional enrichment or purification process.

106. A method of preparing a pharmaceutical composition comprising a nucleic acid, the method comprising:
obtaining a conductivity measurement of a composition comprising the nucleic acid;
when the conductivity measurement is at or below a threshold, formulating the nucleic acid of the composition into the pharmaceutical composition.
